(19) United States
(12) Patent Application Publication
(10) Pub. No.: US 2021/0369250 A1
(43) Pub. Date: Dec. 2, 2021

(54) NON-COVALENT MODIFICATION OF GRAPHENE WITH NANOPARTICLES

(71) Applicants: REGENTS OF THE UNIVERSITY OF MINNESOTA, Minneapolis, MN (US); Boston Scientific Scimed, Inc., Maple Grove, MN (US)

(72) Inventors: Philippe Pierre Joseph Buhlmann, Minneapolis, MN (US); Steven J. Koester, Edina, MN (US); Justin Theodore Nelson, Minneapolis, MN (US); Xue Zhen, Minneapolis, MN (US)

(21) Appl. No.: 17/328,478

(22) Filed: May 24, 2021

Related U.S. Application Data
(60) Provisional application No. 63/030,139, filed on May 26, 2020.

(51) Int. Cl. A61B 10/00; C01B 32/194; C01G 7/00

(52) U.S. Cl. CPC A61B 10/00; B82Y 5/00; C01G 7/00; C01B 32/194

(57) ABSTRACT

Embodiments herein relate to chemical sensors, devices and systems including the same, and related methods. In an embodiment, a medical device is included having a graphene varactor. The graphene varactor includes a graphene layer and at least one non-covalent modification layer disposed on an outer surface of the graphene layer. The non-covalent modification layer can include nanoparticles selected from a group that can include one or more metals, metal oxides, or derivatives thereof. Other embodiments are also included herein.

This application claims the benefit of U.S. Provisional Application No. 63/030,139, filed May 26, 2020, the content of which is herein incorporated by reference in its entirety.

### FIELD

Embodiments herein relate to chemical sensors, devices and systems including the same, and related methods. More specifically, embodiments herein relate to chemical sensors based on the non-covalent surface modification of graphene with nanoparticles.

### BACKGROUND

The accurate detection of diseases can allow clinicians to provide appropriate therapeutic interventions. The early detection of diseases can lead to better treatment outcomes. Diseases can be detected using many different techniques including analyzing tissue samples, analyzing various bodily fluids, diagnostic scans, genetic sequencing, and the like.

Some disease states result in the production of specific chemical compounds. In some cases, volatile organic compounds (VOCs) released into a gaseous sample of a patient can be hallmarks of certain diseases. The detection of these compounds or differential sensing of the same can allow for the early detection of particular disease states.

### SUMMARY

Embodiments herein relate to chemical sensors based on the non-covalent surface modification of graphene with nanoparticles. In a first aspect, a medical device is included having a graphene varactor. The graphene varactor includes a graphene layer and at least one non-covalent modification layer disposed on an outer surface of the graphene layer. The non-covalent modification layer includes nanoparticles selected from a group that can include one or more metals, metal oxides, or derivatives thereof.

In a second aspect, in addition to one or more of the preceding or following aspects, or in the alternative to some aspects, wherein the at least one modification layer provides coverage over the graphene layer from 5% to 150% by surface area.

In a third aspect, in addition to one or more of the preceding or following aspects, or in the alternative to some aspects, the medical device can include a plurality of graphene varactors configured in an array on the medical device.

In a fourth aspect, in addition to one or more of the preceding or following aspects, or in the alternative to some aspects, the medical device further can include more than one non-covalent modification layer.

In a fifth aspect, in addition to one or more of the preceding or following aspects, or in the alternative to some aspects, the medical device can include from two to 20 distinct non-covalent modification layers.

In a sixth aspect, in addition to one or more of the preceding or following aspects, or in the alternative to some aspects, the nanoparticles can include metals or metal oxides selected from a group can include gold (Au), platinum (Pt), silver (Ag), palladium (Pd), iron III oxide (Fe₂O₃), iron II, III oxide (Fe₃O₄), zinc oxide (ZnO), palladium II oxide (PdO), tin dioxide (SnO₂), titanium (Ti), titanium dioxide (TiO₂), silicon dioxide (SiO₂), cobalt diiron tetraoxide (CoFe₂O₄), indium trioxide (In₂O₃), vanadium pentoxide (V₂O₅), platinum oxide (PtO₂), copper oxide (CuO), cadmium oxide (CdO), chromium niobate (CrNbO₄), CoNb₂O₆, molybdenum disulfide (MoS₂), tungsten oxide (WO), tungsten dioxide (WO₂), tungsten trioxide (WO₃), neodymium oxide (Nd₂O₃), boron nitride (BN), CeFeO₄H, manganese oxide (Mn₃O₄), iron oxyhydroxide (FeOOH) can include goethite, akageneite, lepidocrocite, and feroxyhyte, and any combinations, or derivatives thereof. In a seventh aspect, in addition to one or more of the preceding or following aspects, or in the alternative to some aspects, wherein the nanoparticles include gold nanoparticles.

In an eighth aspect, in addition to one or more of the preceding or following aspects, or in the alternative to some aspects, wherein the nanoparticles are further modified with groups that can include alkylthio, alkenylthio, alkynylthio, heteroalkylthio, heteroalkenylthio, heteroalkynylthio, haloalkylthio, haloalkenylthio, haloalkynylthio, halogenated heteroalkylthio, halogenated heteroalkenylthio, halogenated heteroalkynylthio, arylthio, substituted arylthio, heteroarylthio, or substituted heteroarylthio.

In a ninth aspect, a method of modifying a surface of graphene is included. The method can include contacting a graphene layer with a solution or suspension including one or more nanoparticles including metals, metal oxides, or derivatives thereof. The method can include forming at least one non-covalent modification layer of the one or more nanoparticles disposed on an outer surface of a graphene layer, wherein the at least one non-covalent modification layer includes one or more nanoparticles selected from a group can include one or more metals, metal oxides, or derivatives thereof. The method can include quantifying an extent of surface coverage of the at least one non-covalent modification layer using contact angle goniometry, Raman spectroscopy, scanning electron microscopy (SEM), transmission electron microscopy (TEM), atomic force microscopy (AFM), scanning tunneling microscopy (STM), or X-Ray photoelectron spectroscopy.

In a tenth aspect, in addition to one or more of the preceding or following aspects, or in the alternative to some aspects, where the at least one non-covalent modification layer provides coverage over the graphene layer from 5% to 150% by surface area. In an eleventh aspect, in addition to one or more of the preceding or following aspects, or in the alternative to some aspects, wherein contacting a graphene layer with a solution or a suspension includes immersing the graphene layer into a solution or a suspension.

In a twelfth aspect, in addition to one or more of the preceding or following aspects, or in the alternative to some aspects, the method further can include forming more than one non-covalent modification layer.

In a thirteenth aspect, in addition to one or more of the preceding or following aspects, or in the alternative to some aspects, the nanoparticles can include metals or metal oxides selected from a group can include gold (Au), platinum (Pt), silver (Ag), palladium (Pd), iron III oxide (Fe₂O₃), iron II, III oxide (Fe₃O₄), zinc oxide (ZnO), palladium II oxide (PdO), tin dioxide (SnO₂), titanium (Ti), titanium dioxide (TiO₂), silicon dioxide (SiO₂), cobalt diiron tetraoxide (CoFe₂O₄), indium trioxide (In₂O₃), vanadium pentoxide (V₂O₅), platinum oxide (PtO₂), copper oxide (CuO), cadmium oxide (CdO), chromium niobate (CrNbO₄), CoNb₂O₆, molybdenum disulfide (MoS₂), tungsten oxide (WO), tungsten dioxide (WO₂), tungsten trioxide (WO₃), neodymium oxide (Nd₂O₃), boron nitride (BN), CeFeO₄H, manganese oxide (Mn₃O₄), iron oxyhydroxide (FeOOH) can include goethite, akageneite, lepidocrocite, and feroxyhyte, and any combinations, or derivatives thereof.

In a fourteenth aspect, in addition to one or more of the preceding or following aspects, or in the alternative to some aspects, wherein the nanoparticles are further modified with groups can include alkylthio, alkenylthio, alkynylthio, heteroalkylthio, heteroalkenylthio, heteroalkynylthio, haloalkylthio, haloalkenylthio, haloalkynylthio, halogenated heteroalkylthio, halogenated heteroalkenylthio, halogenated heteroalkynylthio, arylthio, substituted arylthio, heteroarylthio, or substituted heteroarylthio.

In a fifteenth aspect, a method for detecting an analyte is included. The method can include collecting a gaseous sample and contacting the gaseous sample with one or more graphene varactors, where each of the one or more graphene varactors includes a graphene layer and, at least one non-covalent modification layer disposed on an outer surface of the graphene layer. The non-covalent modification layer includes one or more nanoparticles selected from a group can include metals, metal oxides, or derivatives thereof.

In a sixteenth aspect, in addition to one or more of the preceding or following aspects, or in the alternative to some aspects, the gaseous sample can include a patient breath sample or an environmental gas sample.

In a seventeenth aspect, in addition to one or more of the preceding or following aspects, or in the alternative to some aspects, the method further can include measuring a differential response in an electrical property of the one or more graphene varactors due to binding of one or more analytes present in the gaseous sample.

In an eighteenth aspect, in addition to one or more of the preceding or following aspects, or in the alternative to some aspects, the electrical property can be selected from the group consisting of capacitance or resistance.

In a nineteenth aspect, in addition to one or more of the preceding or following aspects, or in the alternative to some aspects, the nanoparticles can include metals or metal oxides selected from the group can include gold (Au), platinum (Pt), silver (Ag), palladium (Pd), iron III oxide (Fe₂O₃), iron II, III oxide (Fe₃O₄), zinc oxide (ZnO), palladium II oxide (PdO), tin dioxide (SnO₂), titanium (Ti), titanium dioxide (TiO₂), silicon dioxide (SiO₂), cobalt diiron tetraoxide (CoFe₂O₄), indium trioxide (In₂O₃), vanadium pentoxide (V₂O₅), platinum oxide (PtO₂), copper oxide (CuO), cadmium oxide (CdO), chromium niobate (CrNbO₄), CoNb₂O₆, molybdenum disulfide (MoS₂), tungsten oxide (WO), tungsten dioxide (WO₂), tungsten trioxide (WO₃), neodymium oxide (Nd₂O₃), boron nitride (BN), CeFeO₄H, manganese oxide (Mn₃O₄), iron oxyhydroxide (FeOOH) can include goethite, akageneite, lepidocrocite, and feroxyhyte, and any combinations, or derivatives thereof.

In a twentieth aspect, in addition to one or more of the preceding or following aspects, or in the alternative to some aspects, wherein the nanoparticles are further modified with groups can include alkylthio, alkenylthio, alkynylthio, heteroalkylthio, heteroalkenylthio, heteroalkynylthio, haloalkylthio, haloalkenylthio, haloalkynylthio, halogenated heteroalkylthio, halogenated heteroalkenylthio, halogenated heteroalkynylthio, arylthio, substituted arylthio, heteroarylthio, or substituted heteroarylthio.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic perspective view of a graphene varactor in accordance with various embodiments herein.

FIG. 2 is a schematic cross-sectional view of a portion of a graphene varactor in accordance with various embodiments herein.

FIG. 3 is a schematic top plan view of a chemical sensor element in accordance with various embodiments herein.

FIG. 4 is a schematic diagram of a portion of a measurement zone in accordance with various embodiments herein.

FIG. 5 is a circuit diagram of a passive sensor circuit and a portion of a reading circuit in accordance with various embodiments herein.

FIG. 6 is a schematic diagram of circuitry to measure the capacitance of a plurality of discrete graphene varactors in accordance with various embodiments herein.

FIG. 7 is a schematic view of a system for sensing gaseous analytes in accordance with various embodiments herein.

FIG. 8 is a schematic view of a system for sensing gaseous analytes in accordance with various embodiments herein.

FIG. 9 is a schematic cross-sectional view of a portion of a chemical sensor element in accordance with various embodiments herein.

FIG. 10 is a graph showing capacitance versus DC bias voltage for a graphene varactor in accordance with various embodiments herein.

FIG. 11 is a graph showing capacitance versus DC bias voltage for a graphene varactor in accordance with various embodiments herein.

FIG. 12 is a representative plot of capacitance versus DC bias voltage for a graphene varactor in accordance with various embodiments herein.

FIG. 13 is a representative plot of capacitance versus DC bias voltage for a graphene varactor in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Embodiments herein relate to chemical sensors, medical devices, and systems including the same, and related methods for detecting chemical compounds and elemental molecules in gaseous samples, such as, but not limited to, the breath of a patient. In some embodiments, the chemical sensors herein can be based on the non-covalent surface modification of graphene with nanoparticles.

Chemical sensors having one or more discrete binding detectors can be configured to bind one or more analytes, such as volatile organic compounds (VOCs), in a complex gaseous mixture, such as breath. The discrete binding detectors can include graphene quantum capacitance varactors (“graphene varactors”) that can exhibit a change in capacitance in response to an applied bias voltage as a result of the presence of one or more analytes, such as volatile organic compounds (VOCs) on a surface of the graphene varactor. In this way, gas samples can be analyzed by contacting them with a graphene varactor-based sensor element, providing a bias voltage, and measuring capacitance.

As used herein, the term “analyte” can include various molecular compounds such as volatile organic compounds and elemental molecules such as oxygen. In some cases, analytes can be indicative of various disease states in a patient, various healthy states within a patient, or of pharmaceutical metabolites.

Graphene is a form of carbon containing a single layer of carbon atoms in a hexagonal lattice. Graphene has a high strength and stability due to its tightly packed sp² hybridized orbitals, where each carbon atom forms one sigma (a) bond each with its three neighboring carbon atoms and has one p orbital projected out of the hexagonal plane. The p orbitals of the hexagonal lattice can hybridize to form a 7C band on the surface of graphene that is suitable for non-covalent electrostatic interactions, including π-π stacking interactions with other molecules.

Nanoparticles are particles that exist on a nanometer scale. They lend themselves to chemical sensing at least partially due to their large surface area to volume ratio and unique interactions to analytes of interest. In the embodiments herein, various nanoparticles, including those such as gold nanoparticles and 1-octanethiol functionalized gold nanoparticles, are described. Nanoparticles can be functionalized with various groups to attract various analytes of interest and to provide binding diversity within a given population of nanoparticles.

Nanoparticles can be deposited onto graphene through non-covalent interactions such as electrostatic interactions and Van der Waals interactions. In various embodiments, non-functionalized nanoparticles can be deposited onto graphene, while in other embodiments functionalized nanoparticles can be deposited onto graphene. In yet other embodiments, mixtures of non-functionalized and functionalized nanoparticles can be deposited onto graphene. In some embodiments, the nanoparticles herein can be modified covalently with various groups. In some cases, the nanoparticles can be covalently modified with groups that interact non-covalently with the surface of a graphene layer. Groups that interact non-covalently with the surface of a graphene layer can include those directly in contact with the surface of the graphene layer or those that are in peripheral contact with the surface of the graphene layer. In other embodiments, the nanoparticles can be covalently modified with groups that have binding specificity for various analytes.

The presence of a layer of nanoparticles on graphene can be characterized by various techniques, including the use of X-ray photoelectron spectroscopy (XPS). Capacitance-voltage measurements can also be performed on graphene-based varactors to measure how the Dirac point of a graphene varactor can shift after nanoparticle functionalization.

The graphene varactor-based sensor elements can be exposed to a range of bias voltages in order to discern features such as the Dirac point (or the bias voltage at which the varactor exhibits the lowest capacitance). The response signal generated by the discrete binding detectors in the presence or absence of one or more analytes can be used to characterize the functionalization of a graphene surface, and can further be used to characterize the content of a gaseous mixture.

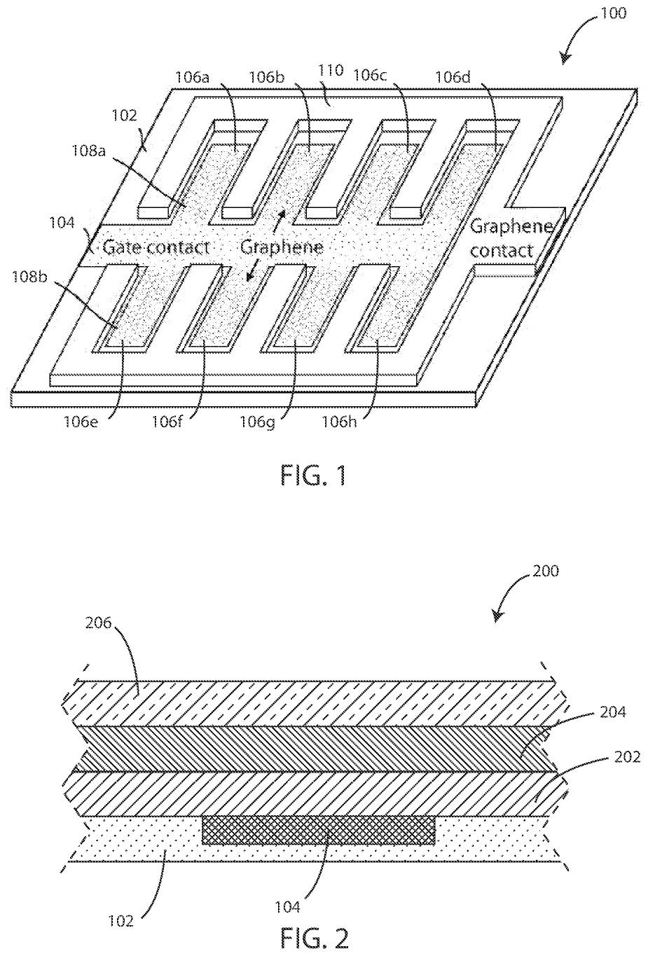

Referring now to FIG. 1, a schematic view of a graphene-based variable capacitor (or graphene varactor) 100 is shown in accordance with the embodiments herein. It will be appreciated that graphene varactors can be prepared in various ways with various geometries, and that the graphene varactor shown in FIG. 1 is just one example in accordance with the embodiments herein.

Graphene varactor 100 can include an insulator layer 102, a gate electrode 104 (or “gate contact”), a dielectric layer (not shown in FIG. 1), one or more graphene layers, such as graphene layers 108a and 108b, and a contact electrode 110 (or “graphene contact”). In some embodiments, the graphene layer(s) 108a-b can be contiguous, while in other embodiments the graphene layer(s) 108a-b can be non-contiguous. Gate electrode 104 can be deposited within one or more depressions formed in insulator layer 102. Insulator layer 102 can be formed from an insulative material such as silicon dioxide, formed on a silicon substrate (wafer), and the like. Gate electrode 104 can be formed by an electrically conductive material such as chromium, copper, gold, silver, nickel, tungsten, aluminum, titanium, palladium, platinum, iridium, and any combinations or alloys thereof, which can be deposited on top of or embedded within the insulator layer 102. The dielectric layer can be disposed on a surface of the insulator layer 102 and the gate electrode 104. The graphene layer(s) 108a-b can be disposed on the dielectric layer. The dielectric layer will be discussed in more detail below in reference to FIG. 2.

Graphene varactor 100 includes eight gate electrode fingers 106a-106h. It will be appreciated that while graphene varactor 100 shows eight gate electrode fingers 106a-106h, any number of gate electrode finger configurations can be contemplated. In some embodiments, an individual graphene varactor can include fewer than eight gate electrode fingers. In some embodiments, an individual graphene varactor can include more than eight gate electrode fingers. In other embodiments, an individual graphene varactor can include two gate electrode fingers. In some embodiments, an individual graphene varactor can include 1, 2, 3, 4, 5, 6, 7, 8, 9, 10, or more gate electrode fingers.

Graphene varactor 100 can include one or more contact electrodes 110 disposed on portions of the graphene layers 108a and 108b. Contact electrode 110 can be formed from an electrically conductive material such as chromium, copper, gold, silver, nickel, tungsten, aluminum, titanium, palladium, platinum, iridium, and any combinations or alloys thereof. Further aspects of exemplary graphene varactors can be found in U.S. Pat. No. 9,513,244, the content of which is herein incorporated by reference in its entirety.

The graphene varactors described herein can include those in which a single graphene layer has been surface-modified through non-covalent interactions between graphene and one or more nanoparticles, such as various metals and metal oxides, or derivatives thereof. In some embodiments, the nanoparticles can include gold (Au) nanoparticles. In some embodiments, the nanoparticles can include one or more of gold (Au), platinum (Pt), silver (Ag), palladium (Pd), iron III oxide (Fe₂O₃), iron II, III oxide (Fe₃O₄), zinc oxide (ZnO), palladium II oxide (PdO), tin dioxide (SnO₂), titanium (Ti), titanium dioxide (TiO₂), silicon dioxide (SiO₂), cobalt diiron tetraoxide (CoFe₂O₄), indium trioxide (In₂O₃), vanadium pentoxide (V₂O₅), platinum oxide (PtO₂), copper oxide (CuO), cadmium oxide (CdO), chromium niobate (CrNbO₄), CoNb₂O₆, molybdenum disulfide (MoS₂), tungsten oxide (WO), tungsten dioxide (WO₂), tungsten trioxide (WO₃), neodymium oxide (Nd₂O₃), boron nitride (BN), CeFeO₄H, manganese oxide (Mn₃O₄), any form of iron oxyhydroxide (FeOOH, which includes goethite, akageneite, lepidocrocite, and feroxyhyte, or any other form of FeOOH), or any combinations or derivatives thereof.

In various embodiments, the nanoparticles herein can include modifications such as with alkylthio, alkenylthio, alkynylthio, heteroalkylthio, heteroalkenylthio, heteroalkynylthio, haloalkylthio, haloalkenylthio, haloalkynylthio, halogenated heteroalkylthio, halogenated heteroalkenylthio, halogenated heteroalkynylthio, arylthio, substituted arylthio, heteroarylthio, or substituted heteroarylthio groups as described herein.

As used herein, the term “alkyl” refers to any linear, branched, or cyclic hydrocarbon group containing anywhere from 1 to 20 carbon atoms (i.e., C₁-C₂₀ alkyl). In some embodiments, the alkyl groups herein can contain any linear, branched, or cyclic hydrocarbon group containing anywhere from 6 to 18 carbon atoms (i.e., C₆-C₁₈ alkyl). In other embodiments, the alkyl groups herein can contain any linear, branched, or cyclic hydrocarbon group containing anywhere from 10 to 16 carbon atoms (i.e., C₁₀-C₁₆ alkyl). The alkyl groups described herein have the general formula CₙH₂ₙ₊₁, unless otherwise indicated.

As used herein, the term “alkylthio” refers to a group having the general formula R—S— where R is an alkyl group as defined herein that is covalently bonded to a sulfur atom.

As used herein, the term “alkenyl” refers to any linear, branched, or cyclic hydrocarbon group containing anywhere from 1 to 20 carbon atoms, wherein the alkenyl group contains at least one carbon-carbon double bond (i.e., C₁-C₂₀ alkenyl). In some embodiments, the alkenyl groups herein can contain any linear, branched, or cyclic hydrocarbon group containing anywhere from 6 to 18 carbon atoms, wherein the alkenyl group contains at least one carbon-carbon double bond (i.e., C₆-C₁₈ alkenyl). In other embodiments, the alkenyl groups herein can contain any linear, branched, or cyclic hydrocarbon group containing anywhere from 10 to 16 carbon atoms, wherein the alkenyl group contains at least one carbon-carbon double bond (i.e., C₁₀-C₁₆ alkenyl). The alkenyl groups described herein have the general formula CₙH₍₂ₙ₊₁₋₂ₓ₎, where x is the number of double bonds present in the alkenyl group, unless otherwise indicated.

As used herein, the term “alkenylthio” refers to a group having the general formula R—S— where R is an alkenyl group as defined herein that is covalently bonded to a sulfur atom.

As used herein, the term “alkynyl” refers to any linear, branched, or cyclic hydrocarbon group containing anywhere from 1 to 20 carbon atoms, including one or more carbon-carbon triple bonds (i.e., C₁-C₂₀ alkynyl). In some embodiments, the alkynyl groups herein can contain any linear, branched, or cyclic hydrocarbon group containing anywhere from 6 to 18 carbon atoms, including one or more carbon-carbon triple bonds (i.e., C₆-C₁₈ alkynyl). In other embodiments, the alkynyl groups herein can contain any linear, branched, or cyclic hydrocarbon group containing anywhere from 10 to 16 carbon atoms, including one or more carbon-carbon triple bonds (i.e., C₁₀-C₁₆ alkynyl).

As used herein, the term “alkynylthio” refers to a group having the general formula R—S— where R is an alkynyl group as defined herein that is covalently bonded to a sulfur atom.

As used herein, the term “heteroalkyl” refers to any linear, branched, or cyclic hydrocarbon group containing anywhere from 1 to 20 carbon atoms, and one or more heteroatoms, including, but not limited to, N, O, P, S, Si, Se, and B, or any combination thereof (i.e., C₁-C₂₀ heteroalkyl). In some embodiments, the heteroalkyl groups herein can contain any linear, branched, or cyclic hydrocarbon group containing anywhere from 6 to 18 carbon atoms and one or more heteroatoms, including, but not limited to, N, O, P, S, Si, Se, and B, or any combination thereof (i.e., C₆-C₁₈ heteroalkyl). In other embodiments, the heteroalkyl groups herein can contain any linear, branched, or cyclic hydrocarbon group containing anywhere from 10 to 16 carbon atoms and one or more heteroatoms, including, but not limited to, N, O, P, S, Si, Se, and B, or any combination thereof (i.e., C₁₀-C₁₆ heteroalkyl). In some embodiments, the heteroalkyl groups herein can have the general formula —RZ, —RZR, —ZRZR, or —RZRZR, where R can include, but is not to be limited to, any identical or different, linear, branched, or cyclic, C₁-C₂₀ alkyl, or a combination thereof; and Z can include one or more heteroatoms including, but is not limited to, N, O, P, S, Si, Se, and B, or any combination thereof.

In some embodiments, the heteroalkyl group can include, but is not to be limited to, alkoxy groups, alkyl amide groups, alkyl thioether groups, alkyl ester groups, alkyl sulfonate groups, alkyl phosphate groups, and the like. Examples of heteroalkyl groups suitable for use herein can include, but is not to be limited to, those selected from —ROH, —RC(O)OH, —RC(O)OR, —ROR, —RSR, —RCHO, —RX, —RC(O)NH₂, —RC(O)NR, —RNH₃⁺, —RNH₂, —RNO₂, —RNHR, —RNRR, —RB(OH)₂, —RSO₃⁻, —RPO₄²⁻, or any combination thereof; where R can include, but is not to be limited to, any identical or different, linear, branched, or cyclic, C₁-C₂₀ alkyl, C₁-C₂₀ heteroalkyl, provided that at least one heteroatom including, but not limited to, N, O, P, S, Si, Se, and B, is present in at least one R group, or a combination thereof; and X can be a halogen including F, Cl, Br, I, or At.

As used herein, the term “heteroalkylthio” refers to a group having the general formula R—S— where R is a heteroalkyl group as defined herein that is covalently bonded to a sulfur atom.

As used herein, the term “heteroalkenyl” refers to any linear, branched, or cyclic hydrocarbon group containing anywhere from 1 to 20 carbon atoms, including one or more carbon-carbon double bonds, and one or more heteroatoms including, but not limited to, N, O, P, S, Si, Se, and B, or any combination thereof (i.e., C₁-C₂₀ heteroalkenyl). In some embodiments, the heteroalkenyl groups herein can contain any linear, branched, or cyclic hydrocarbon group containing anywhere from 6 to 18 carbon atoms, including one or more carbon-carbon double bonds, and one or more heteroatoms, including, but not limited to, N, O, P, S, Si, Se, and B, or any combination thereof (i.e., C₆-C₁₈ heteroalkenyl). In other embodiments, the heteroalkenyl groups herein can contain any linear, branched, or cyclic hydrocarbon group containing anywhere from 10 to 16 carbon atoms, including one or more carbon-carbon double bonds, and one or more heteroatoms, including, but not limited to, N, O, P, S, Si, Se, and B, or any combination thereof (i.e., C₁₀-C₁₆ heteroalkenyl). In some embodiments, the heteroalkenyl groups herein can have the general formula —RZ, —RZR, —ZRZR, or —RZRZR, where R can include, but is not to be limited to, any identical or different, linear, branched, or cyclic, C₁-C₂₀ alkyl or C₁-C₂₀ alkenyl, provided that at least one carbon-carbon double bond is present in at least one R group, or a combination thereof; and Z can include one or more heteroatoms including, but not limited to, N, O, P, S, Si, Se, and B, or any combination thereof.

In some embodiments, the heteroalkenyl group can include, but is not to be limited to, alkenoxy groups, alkenyl amines, alkenyl thioester groups, alkenyl ester groups, alkenyl sulfonate groups, alkenyl phosphate groups, and the like. Examples of heteroalkenyl groups suitable for use herein can include, but is not to be limited to, those selected from —ROH, —RC(O)OH, —RC(O)OR, —ROR, —RSR, —RCHO, —RX, —RC(O)NH₂, —RC(O)NR, —RNH₃⁺, —RNH₂, —RNO₂, —RNHR, —RNRR, —RB(OH)₂, —RSO₃⁻, —RPO₄²⁻, or any combination thereof; where R can include, but is not to be limited to, any identical or different, linear, branched, or cyclic, C₁-C₂₀ alkyl, or C₁-C₂₀ alkenyl, provided that at least one or more carbon-carbon double bonds and one or more heteroatoms including, but not limited to, N, O, P, S, Si, Se, and B, or any combination thereof, are present in at least one R group; or a combination thereof; and X can be a halogen including F, Cl, Br, I, or At.

As used herein, the term “heteroalkenylthio” refers to a group having the general formula R—S— where R is a heteroalkenyl group as defined herein that is covalently bonded to a sulfur atom.

As used herein, the term “heteroalkynyl” refers to any linear, branched, or cyclic hydrocarbon group containing anywhere from 1 to 20 carbon atoms, including one or more carbon-carbon triple bonds, and one or more heteroatoms including, but not limited to, N, O, P, S, Si, Se, and B, or any combination thereof (i.e., C₁-C₂₀ heteroalkynyl). In some embodiments, the heteroalkynyl groups herein can contain any linear, branched, or cyclic hydrocarbon group containing anywhere from 6 to 18 carbon atoms, including one or more carbon-carbon triple bonds, and one or more heteroatoms, including, but not limited to, N, O, P, S, Si, Se, and B, or any combination thereof (i.e., C₆-C₁₈ heteroalkynyl). In other embodiments, the heteroalkynyl groups herein can contain any linear, branched, or cyclic hydrocarbon group containing anywhere from 10 to 16 carbon atoms, including one or more carbon-carbon triple bonds, and one or more heteroatoms, including, but not limited to, N, O, P, S, Si, Se, and B, or any combination thereof (i.e., C₁₀-C₁₆ heteroalkynyl). In some embodiments, the heteroalkynyl groups herein can have the general formula —RZ, —RZR, —ZRZR, or —RZRZR, where R can include, but is not to be limited to, any identical or different, linear, branched, or cyclic, C₁-C₂₀ alkyl, C₁-C₂₀ alkenyl, or C₁-C₂₀ alkynyl, provided that at least one carbon-carbon triple bond is present in at least one R group or a combination thereof; and Z can include one or more heteroatoms including, but not limited to, N, O, P, S, Si, Se, and B, or any combination thereof.

In some embodiments, the heteroalkynyl group can include, but is not to be limited to, alkynyloxy groups, alkynyl amines, alkynyl thioester groups, alkynyl ester groups, alkenyl sulfonate groups, alkenyl phosphate groups, and the like. Examples of heteroalkynyl groups suitable for use herein can include, but is not to be limited to, those selected from —ROH, —RC(O)OH, —RC(O)OR, —ROR, —RSR, —RCHO, —RX, —RC(O)NH₂, —RC(O)NR, —RNH₃⁺, —RNH₂, —RNO₂, —RNHR, —RNRR, —RB(OH)₂, —RSO₃⁻, —RPO₄²⁻, or any combination thereof; where R can include, but is not to be limited to, any identical or different, linear, branched, or cyclic, C₁-C₂₀ alkyl, C₁-C₂₀ alkenyl, or C₁-C₂₀ alkynyl, provided that at least one or more carbon-carbon triple bonds and one or more heteroatoms including, but not limited to, N, O, P, S, Si, Se, and B, or any combination thereof, are present in at least one R group; or a combination thereof; and X can be a halogen including F, Cl, Br, I, or At.

As used herein, the term “heteroalkynylthio” refers to a group having the general formula R—S— where R is a heteroalkynyl group as defined herein that is covalently bonded to a sulfur atom.

As used herein, the term “haloalkyl” refers to any linear, branched, or cyclic alkyl groups containing anywhere from 1 to 20 carbon atoms (i.e., C₁-C₂₀) having one or more hydrogen atoms replaced by a halogen atom including at least one of F, Cl, Br, I, At (i.e., C₁-C₂₀ haloalkyl). In some embodiments, the haloalkyl groups herein can contain any linear, branched, or cyclic alkyl group containing anywhere from 6 to 18 carbon atoms having one or more hydrogen atoms replaced by a halogen atom including at least one of F, Cl, Br, I, or At (i.e., C₆-C₁₈ haloalkyl). In other embodiments, the haloalkyl groups herein can contain any linear, branched, or cyclic alkyl group containing anywhere from 10 to 16 carbon atoms having one or more hydrogen atoms replaced by a halogen atom including at least one of F, Cl, Br, I, or At (i.e., C₁₀-C₁₆ haloalkyl). In some embodiments, the haloalkyl can include a monohaloalkyl containing only one halogen atom in place of a hydrogen atom. In other embodiments, the haloalkyl can include a polyhaloalkyl containing more than one halogen atom in place of a hydrogen atom, provided at least one hydrogen atom remains. In yet other embodiments, the haloalkyl can include a perhaloalkyl containing a halogen atom in place of every hydrogen atom of the corresponding alkyl.

As used herein, the term “haloalkylthio” refers to a group having the general formula R—S— where R is a haloalkyl group as defined herein that is covalently bonded to a sulfur atom.

As used herein, the term “haloalkenyl” refers to any linear, branched, or cyclic alkenyl group containing anywhere from 1 to 20 carbon atoms (i.e., C₁-C₂₀) having one or more hydrogen atoms replaced by a halogen atom including at least one of F, Cl, Br, I, or At, and wherein the haloalkenyl group contains at least one carbon-carbon double bond (i.e., C₁-C₂₀ haloalkenyl). In some embodiments, the haloalkenyl groups herein can contain any linear, branched, or cyclic alkenyl group containing anywhere from 6 to 18 carbon atoms, having one or more hydrogen atoms replaced by a halogen atom including at least one of F, Cl, Br, I, or At, and wherein the haloalkenyl group contains at least one carbon-carbon double bond (i.e., C₆-C₁₈ haloalkenyl). In other embodiments, the haloalkenyl groups herein can contain any linear, branched, or cyclic alkenyl group containing anywhere from 10 to 16 carbon atoms, having one or more hydrogen atoms replaced by a halogen atom including at least one of F, Cl, Br, I, or At, and wherein the haloalkenyl group contains at least one carbon-carbon double bond (i.e., C₁₀-C₁₆ haloalkenyl).

As used herein, the term “haloalkenylthio” refers to a group having the general formula R—S— where R is a haloalkenyl group as defined herein that is covalently bonded to a sulfur atom.

As used herein, the term “haloalkynyl” refers to any linear, branched, or cyclic alkynyl group containing anywhere from 1 to 20 carbon atoms (i.e., C₁-C₂₀) having one or more hydrogen atoms replaced by a halogen atom including at least one of F, Cl, Br, I, or At, and wherein the haloalkynyl group contains at least one carbon-carbon triple bond (i.e., C₁-C₂₀ haloalkynyl). In some embodiments, the haloalkynyl groups herein can contain any linear, branched, or cyclic alkynyl group containing anywhere from 6 to 18 carbon atoms, having one or more hydrogen atoms replaced by a halogen atom including at least one of F, Cl, Br, I, or At, and wherein the haloalkynyl group contains one or more carbon-carbon triple bonds (i.e., C₆-C₁₈ haloalkynyl). In other embodiments, the haloalkynyl groups herein can contain any linear, branched, or cyclic alkynyl group containing anywhere from 10 to 16 carbon atoms, having one or more hydrogen atoms replaced by a halogen atom including at least one of F, Cl, Br, I, or At, and wherein the haloalkynyl group contains one or more carbon-carbon triple bonds (i.e., C₁₀-C₁₆ haloalkynyl).

As used herein, the term “haloalkynylthio” refers to a group having the general formula R—S— where R is a haloalkynyl group as defined herein that is covalently bonded to a sulfur atom.

As used herein, the term “halogenated heteroalkyl” refers to any heteroalkyl group as described herein, containing anywhere from 1 to 20 carbon atoms (i.e., C₁-C₂₀) and having one or more hydrogen atoms replaced by a halogen atom, including at least one of F, Cl, Br, I, or At (i.e., C₁-C₂₀ halogenated heteroalkyl). In some embodiments, the halogenated heteroalkyl groups herein can include any heteroalkyl group as described herein, containing anywhere from 6 to 18 carbon atoms (i.e., C₆-C₁₈) and having one or more hydrogen atoms replaced by a halogen atom, including at least one of F, Cl, Br, I, or At (i.e., C₆-C₁₈ halogenated heteroalkyl). In other embodiments, the halogenated heteroalkyl groups herein can include any heteroalkyl group as described herein, containing anywhere from 10 to 16 carbon atoms (i.e., C₁₀-C₁₆) and having one or more hydrogen atoms replaced by a halogen atom, including at least one of F, Cl, Br, I, or At (i.e., C₁₀-C₁₆) halogenated heteroalkyl).

As used herein, the term “halogenated heteroalkylthio” refers to a group having the general formula R—S— where R is a halogenated heteroalkyl group as defined herein that is covalently bonded to a sulfur atom.

As used herein, the term “halogenated heteroalkenyl” refers to any heteroalkenyl group as described herein, containing anywhere from 1 to 20 carbon atoms (i.e., C₁-C₂₀) and having one or more hydrogen atoms replaced by a halogen atom, including at least one of F, Cl, Br, I, or At (i.e., C₁-C₂₀ halogenated heteroalkenyl). In some embodiments, the halogenated heteroalkenyl groups herein can include any heteroalkenyl group as described herein, containing anywhere from 6 to 18 carbon atoms (i.e., C₆-C₁₈) and having one or more hydrogen atoms replaced by a halogen atom, including at least one of F, Cl, Br, I, or At (i.e., C₆-C₁₈ halogenated heteroalkenyl). In other embodiments, the halogenated heteroalkenyl groups herein can include any heteroalkenyl group as described herein, containing anywhere from 10 to 16 carbon atoms (i.e., C₁₀-C₁₆) and having one or more hydrogen atoms replaced by a halogen atom, including at least one of F, Cl, Br, I, or At (i.e., C₁₀-C₁₆) halogenated heteroalkenyl).

As used herein, the term “halogenated heteroalkenylthio” refers to a group having the general formula R—S— where R is a halogenated heteroalkenyl group as defined herein that is covalently bonded to a sulfur atom.

As used herein, the term “halogenated heteroalkynyl” refers to any heteroalkynyl group as described herein, containing anywhere from 1 to 20 carbon atoms (i.e., C₁-C₂₀) and having one or more hydrogen atoms replaced by a halogen atom, including at least one of F, Cl, Br, I, or At (i.e., C₁-C₂₀ halogenated heteroalkynyl). In some embodiments, the halogenated heteroalkynyl groups herein can include any heteroalkynyl group as described herein, containing anywhere from 6 to 18 carbon atoms (i.e., C₆-C₁₈) and having one or more hydrogen atoms replaced by a halogen atom, including at least one of F, Cl, Br, I, or At (i.e., C₆-C₁₈ halogenated heteroalkynyl). In other embodiments, the halogenated heteroalkynyl groups herein can include any heteroalkynyl group as described herein, containing anywhere from 10 to 16 carbon atoms (i.e., C₁₀-C₁₆) and having one or more hydrogen atoms replaced by a halogen atom, including at least one of F, Cl, Br, I, or At (i.e., C₁₀-C₁₆) halogenated heteroalkynyl).

As used herein, the term “halogenated heteroalkynylthio” refers to a group having the general formula R—S— where R is a halogenated heteroalkynyl group as defined herein that is covalently bonded to a sulfur atom.

As used herein, the term “aryl” refers to any aromatic hydrocarbon group containing a C₅- to C₈-membered aromatic ring, such as, for example, cyclopentadiene, benzene, and derivatives thereof. The corresponding aromatic radicals to the examples provided include, for example, cyclopentadienyl and phenyl radicals, and derivatives thereof. In some embodiments, the aryl groups herein can be further substituted to form substituted aryl groups. As used herein, the term “substituted aryl” refers to any aromatic hydrocarbon group containing a C₅- to C₈-membered aromatic ring, which itself can be substituted with one or more alkyl, alkenyl, alkynyl, heteroalkyl, heteroalkenyl, heteroalkynyl, haloalkyl, haloalkenyl, haloalkynyl, halogenated heteroalkyl, halogenated heteroalkenyl, or halogenated heteroalkynyl groups, or any combination thereof, as described herein.

Halogenation of any of the aryl or substituted aryl groups used herein can include those where one or more hydrogen atoms are replaced by a halogen atom, including at least one of F, Cl, Br, I, or At. Additional substitutions of the aryl or substituted aryl groups can include, but is not to be limited to, —OH, —C(O)OH, —C(O)OR, —OR, —SR, —CHO, —C(O)NH₂, —C(O)NR, —NH₃⁺, —NH₂, —NO₂, —NHR, —NRR, —B(OH)₂, —SO₃⁻, —PO₄²⁻ or any combination, where R is alkyl, alkenyl, alkynyl, heteroalkyl, heteroalkenyl, heteroalkynyl, haloalkyl, haloalkenyl, haloalkynyl, halogenated heteroalkyl, halogenated heteroalkenyl, or halogenated heteroalkynyl groups, or any combination thereof. In some embodiments, the halogenated aryl group can include a chlorophenyl group. In other embodiments, the halogenated aryl group can include a perfluorophenyl group.

As used herein, the term “arylthio” refers to a group having the general formula R—S— where R is an aryl group as defined herein that is covalently bonded to a sulfur atom. As used herein, the term “substituted arylthio” refers to a group having the general formula R—S— where R is a substituted aryl group as defined herein that is covalently bonded to a sulfur (S) atom.

In some embodiments, the aryl groups herein can include one or more heteroatoms to form heteroaryl groups. Suitable heteroatoms for use herein can include, but is not to be limited to, N, O, P, S, Si, Se, and B. As used herein, the term “heteroaryl” refers to any aryl group, as defined herein, where one or more carbon atoms of the C₅- to C₈-membered aromatic ring has been replaced with one or more heteroatoms or combinations of heteroatoms. Examples of heteroaryl groups can include, but is not to be limited to radicals of, pyrrole, thiophene, furan, imidazole, pyridine, and pyrimidine. The heteroaryl groups herein can be further substituted to form substituted heteroaryl groups. As used herein, the term “substituted heteroaryl” refers to any heteroaryl group, as described herein, which is further substituted with one or more alkyl, alkenyl, alkynyl, heteroalkyl, heteroalkenyl, heteroalkynyl haloalkyl, haloalkenyl, haloalkynyl, halogenated heteroalkyl, halogenated heteroalkenyl, or halogenated heteroalkynyl groups, or any combination thereof, as described herein.

Halogenation of any of the heteroaryl or substituted heteroaryl groups described herein can include those where one or more hydrogen atoms are replaced by a halogen atom, including at least one of F, Cl, Br, I, or At. Additional substitutions of the heteroaryl or substituted heteroaryl groups can include, but is not to be limited to, —OH, —C(O)OH, —C(O)OR, —OR, —SR, —CHO, —C(O)NH₂, —C(O)NR, —NH₃⁺, —NH₂, —NO₂, —NHR, —NRR, —B(OH)₂, —SO₃⁻, —PO₄²⁻ or any combination, where R is alkyl, alkenyl, alkynyl, heteroalkyl, heteroalkenyl, heteroalkynyl, haloalkyl, haloalkenyl, haloalkynyl, halogenated heteroalkyl, halogenated heteroalkenyl, or halogenated heteroalkynyl groups, or any combination thereof.

As used herein, the term “heteroarylthio” refers to a group having the general formula R—S— where R is a heteroaryl group as defined herein that is covalently bonded to a sulfur atom. As used herein, the term “substituted heteroarylthio” refers to a group having the general formula R—S— where R is a substituted heteroaryl group as defined herein that is covalently bonded to a sulfur atom.

The nanoparticles herein include modifications that result from reacting the nanoparticles with various reagents. In some embodiments, a reagent having the formula HS—R, where R can include any C₁-C₂₀ hydrocarbon or heterohydrocarbon, as described elsewhere herein, including but not to be limited to —RSO₃⁻, —RPO₄²⁻, or any combination thereof; and can be reacted with nanoparticles to yield a product having a covalent bond between the nanoparticle and sulfur atom, as can be described by the general formula R—S-nanoparticle. In some embodiments, a reagent having the formula HS—RX; where R can include any C₁-C₂₀ hydrocarbon or heterohydrocarbon as described elsewhere herein, including but not to be limited to —RSO₃⁻, —RPO₄²⁻, or any combination thereof; and X includes one or more aromatic rings with one or more substitutions as described herein, such as pyrene, phenyl, biphenyl, heteroaromatic rings, and the like; and can be reacted with nanoparticles to yield a product having a covalent bond between the nanoparticle and sulfur atom, as can be described by the general formula XR—S-nanoparticle.

In some embodiments, a reagent having the formula RSSR′ is included, where R and R′ can include any C₁-C₂₀ hydrocarbon or heterohydrocarbon as described elsewhere herein, including but not to be limited to —RSO₃⁻, —RPO₄²⁻, or any combination thereof; and can be reacted with nanoparticles to yield two individual products having a covalent bond between the nanoparticle and sulfur atom, as can be described by the general formula R—S-nanoparticle and R′—S-nanoparticle.

In some embodiments, a reagent having the formula XRSSR′X; where X includes one or more aromatic rings with one or more substitutions as described herein, such as pyrene, phenyl, biphenyl, heteroaromatic rings, and the like; where R and R′ can include any C₁-C₂₀ hydrocarbon or heterohydrocarbon as described elsewhere herein, including but not to be limited to —RSO₃⁻, —RPO₄²⁻, or any combination thereof; and can be reacted with nanoparticles to yield a product having a covalent bond between the nanoparticle and sulfur atom, as can be described by the general formula X—R—S-nanoparticle and X′—R′—S-nanoparticle.

In some embodiments, a reagent having any of the formulas RSiZ₃, RR′ SiZ₂, or RR′R″SiZ is included, where Z includes any alkoxy group such as methoxy or ethoxy or any halogen atom such as F, Cl, Br, I, or At, and R, R′, and R″ include any C₁-C₂₀ hydrocarbon or heterohydrocarbon as described elsewhere herein, including but not to be limited to —RSO₃⁻, —RPO₄²⁻, or any combination thereof; and can be reacted with nanoparticles to yield products having a covalent bond between the nanoparticle and silicon atom, as can be described by the general formulas R—Z₂-Si-nanoparticle, R—R′—Si-nanoparticle, and R—R′—R″—Si-nanoparticle.

In some embodiments, a reagent having any of the formulas XRSiZ₃, (XR)(X′R′)SiZ₂, or (XR)(X′R′)(X″R″)SiZ is included; where X includes one or more aromatic rings with one or more substitutions as described herein, such as pyrene, phenyl, biphenyl, heteroaromatic rings, and the like; where Z includes any alkoxy group such as methoxy or ethoxy or any halogen atom such as F, Cl, Br, I, or At, and R, R′, and R″ include any C₁-C₂₀ hydrocarbon or heterohydrocarbon as described elsewhere herein, including but not to be limited to —RSO₃⁻, —RPO₄²⁻, or any combination thereof; and can be reacted with nanoparticles to yield products having a covalent bond between the nanoparticle and silicon atom, as can be described by the general formulas X—R—Z₂—Si-nanoparticle, (XR)—(X′R′)—Si-nanoparticle, and (XR)—(X′R′)—(X″R″)—Si-nanoparticle.

In some embodiments, a reagent having any of the formulas RZH₂, RR′ZH, or RR′R″Z is included; where Z includes nitrogen (N) or phosphorus (P); and where R, R′, and R″ include any C₁-C₂₀ hydrocarbon or heterohydrocarbon as described elsewhere herein, including but not to be limited to —RSO₃⁻, —RPO₄²⁻, or any combination thereof; and can be reacted with nanoparticles to yield products having a covalent bond between the nanoparticle and silicon atom, as can be described by the general formulas R—Z—H-nanoparticle, R—Z—H₂-nanoparticle, R—R′—Z-nanoparticle, R—R′—ZH-nanoparticle, or R—R′—R″—Z-nanoparticle.

In some embodiments, a reagent having any of the formulas (XR)ZH₂, (XR)(X′R′)ZH and (XR)(X′R′)(X″R″)Z; where X includes one or more aromatic rings with one or more substitutions as described herein, such as pyrene, phenyl, biphenyl, heteroaromatic rings, and the like; where Z includes nitrogen (N) or phosphorus (P); and where R, R′, and R″ include any C₁-C₂₀ hydrocarbon or heterohydrocarbon as described elsewhere herein, including but not to be limited to —RSO₃⁻, —RPO₄²⁻, or any combination thereof; and can be reacted with nanoparticles to yield products having a covalent bond between the nanoparticle and silicon atom, as can be described by the general formulas (XR)—ZH-nanoparticle, (XR)—ZH₂-nanoparticle, (XR)(X′R′)—Z-nanoparticle, (XR)(X′R′)—ZH-nanoparticle, and (XR)(X′R′)(X″R″)—Z-nanoparticle

The nanoparticles suitable for use herein can include those having different sizes within a range of sizes from about 1 nanometer (nm) to about 1000 nm. In some embodiments, the size of the nanoparticles herein can be greater than or equal to 1 nm, 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, 990 nm, or 1000 nm, or can be an amount falling within a range between any of the foregoing.

The nanoparticles herein can include those in solution or suspension (e.g., a dispersion) in solvents such as water, methanol, ethanol, chloroform, dichloromethane, benzene, toluene, water, acetone, acetonitrile, ethylene glycol, ethers, tetrahydrofuran, dimethylformamide, hexane, ethyl acetate, or in a solid form. In various embodiments, the solution or suspension may also contain stabilizer or surfactant, such as phosphate buffered saline (PBS) or citric acid, in order to stabilize the nanoparticles.

In various embodiments herein the nanoparticles suitable for use in modifying a graphene surface can include one or more nanoparticles with unique analyte binding specificity. In some embodiments, a first population of nanoparticles can be used where the entire first population has the same analyte binding specificity. In other embodiments, a mixture of a first population and a second population of nanoparticles can be used, where the first population of nanoparticles has a different analyte binding specificity than the second binding population. In other embodiments, a mixture of a first population of nanoparticles, a second population of nanoparticles, and a third population of nanoparticles can be used, where each of the first, second, and third populations of nanoparticles all have different analyte binding specificities. It will be appreciated that in yet other embodiments, a fourth, fifth, sixth, seventh, eighth, ninth, or tenth population of nanoparticles can be used. In some cases, the analyte binding specificities can be attributed to the type of nanoparticle used and in other embodiments the analyte binding specificities can be attributed to the functionalization of the nanoparticles used herein. As such, binding diversity of a given modified graphene surface can be tuned by varying the type and the density of each nanoparticle deposited on the graphene surface.

It will be appreciated that in various embodiments herein, graphene can be substituted with other similar single-layer or multi-layer structural materials, including for example, borophene, graphite, carbon nanotubes, or other structural analogues of graphene. Borophene is a single layer of boron atoms arranged in various crystalline configurations.

Referring now to FIG. 2, a schematic cross-sectional view of a portion of a graphene varactor 200 is shown in accordance with various embodiments herein. The graphene varactor 200 can include an insulator layer 102 and a gate electrode 104 recessed into the insulator layer 102. The gate electrode 104 can be formed by depositing an electrically conductive material in the depression in the insulator layer 102, as discussed above in reference to FIG. 1. A dielectric layer 202 can be formed on a surface of the insulator layer 102 and the gate electrode 104. In some examples, the dielectric layer 202 can be formed of a material, such as, silicon dioxide, aluminum oxide, hafnium dioxide, zirconium dioxide, hafnium silicate, or zirconium silicate. In some examples, the dielectric layer 202 can include multiple layers of the dielectric materials listed herein. In some embodiments, the dielectric layer 202 can include alternating layers of different dielectric materials. In some embodiments, the dielectric layer 202 can include alternating layers of aluminum oxide and hafnium dioxide.

The graphene varactor 200 can include a single graphene layer 204 that can be disposed on a surface of the dielectric layer 202. The graphene layer 204 can be surface-modified with a non-covalent modification layer 206. The non-covalent modification layer 206 can be formed of one or more types of nanoparticles, or derivatives thereof, disposed on an outer surface of the graphene layer 204 through non-covalent interactions. In some embodiments, the non-covalent modification layer 206 can be formed of one or more nanoparticles modifications as discussed elsewhere herein.

The non-covalent modification layer 206 can provide at least 5% surface coverage (by area) of the graphene layer 204. In some embodiments, the non-covalent modification layer 206 can provide at least 10% surface coverage of the graphene layer 204. In other embodiments, the non-covalent modification layer 206 can provide at least 15% surface coverage of the graphene layer 204. In some embodiments, the non-covalent modification layer can provide at least 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% surface coverage (by area) of the graphene layer. It will be appreciated that the non-covalent modification layer can provide surface coverage falling within a range wherein any of the forgoing percentages can serve as the lower or upper bound of the range, provided that the lower bound of the range is a value less than the upper bound of the range.

In some embodiments, it will be appreciated that nanoparticles can include more than a monolayer, such as a multilayer. Multilayers can be detected and quantified by techniques such as scanning tunneling microscopy (STM) and other scanning probe microscopies. References herein to a percentage of coverage greater than 100% shall refer to the circumstance where a portion of the surface area is covered by more than a monolayer, such as covered by two, three or potentially more layers of the nanoparticle used. Thus, a reference to 105% coverage herein shall indicate that approximately 5% of the surface area includes more than monolayer coverage over the graphene layer. In some embodiments, graphene surfaces can include 101%, 102%, 103%, 104%, 105%, 110%, 120%, 130%, 140%, 150%, or 175% surface coverage of the graphene layer. It will be appreciated that multilayer surface coverage of the graphene layer can fall within a range of surface coverages, wherein any of the forgoing percentages can serve as the lower or upper bound of the range, provided that the lower bound of the range is a value less than the upper bound of the range. For example, ranges of coverage can include, but are not limited to, 5% to 150% by surface area, 80% to 120% by surface area, 90% to 110%, or 99% to 120% by surface area.

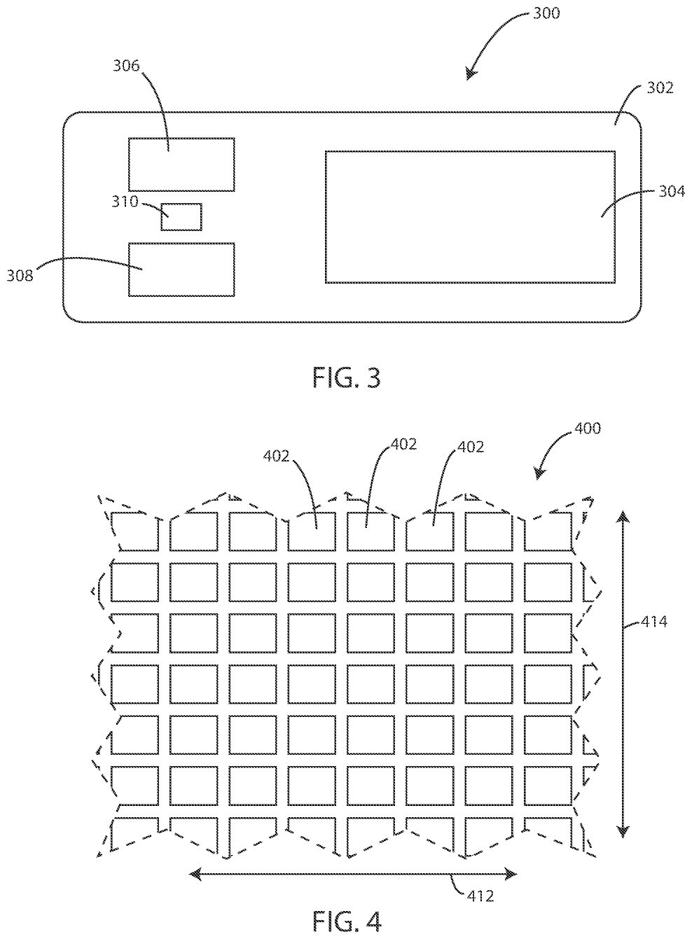

Referring now to FIG. 3, a schematic top plan view of a chemical sensor element 300 is shown in accordance with various embodiments herein. The chemical sensor element can include a graphene varactor-based chemical sensor element. The chemical sensor element 300 can include a substrate 302. It will be appreciated that the substrate can be formed from many different materials. By way of example, the substrate can be formed from silicon, glass, quartz, sapphire, polymers, metals, glasses, ceramics, cellulosic materials, composites, metal oxides, and the like. The thickness of the substrate can vary. In some embodiments, the substrate has sufficient structural integrity to be handled without undue flexure that could damage components thereon. In some embodiments, the substrate can have a thickness of about 0.05 mm to about 5 mm. The length and width of the substrate can also vary. In some embodiments, the length (or major axis) can be from about 0.2 cm to about 10 cm. In some embodiments, the length (or major axis) can be from about 20 μm to about 1 cm. In some embodiments, the width (perpendicular to the major axis) can be from about 0.2 cm to about 8 cm. In some embodiments, the width (perpendicular to the major axis) can be from about 20 μm to about 0.8 cm. In some embodiments, the graphene-based chemical sensor can be disposable.

A first measurement zone 304 can be disposed on the substrate 302. In some embodiments, the first measurement zone 304 can define a portion of a first gas flow path. The first measurement zone (or gas sample zone) 304 can include a plurality of discrete graphene-based variable capacitors (or graphene varactors) that can sense analytes in a gaseous sample, such as a breath sample. In various embodiments, the gaseous sample can include an environmental gas sample. Various gaseous samples are contemplated herein, including gasses released from a variety of bodily tissues, fluids, breath, and the like. Various additional methods for sampling gasses using the chemical sensor elements herein are described in U.S. application Ser. No. 16/696,348, the content of which is herein incorporated by reference.

A second measurement zone (or environment sample zone) 306, separate from the first measurement zone 304, can also be disposed on the substrate 302. The second measurement zone 306 can also include a plurality of discrete graphene varactors. In some embodiments, the second measurement zone 306 can include the same (in type and/or number) discrete graphene varactors that are within the first measurement zone 304. In some embodiments, the second measurement zone 306 can include only a subset of the discrete graphene varactors that are within the first measurement zone 304. In operation, the data gathered from the first measurement zone, which can be reflective of the gaseous sample analyzed, can be corrected or normalized based on the data gathered from the second measurement zone, which can be reflective of analytes present in the environment.

In some embodiments, a third measurement zone (drift control or witness zone) 308 can also be disposed on the substrate. The third measurement zone 308 can include a plurality of discrete graphene varactors. In some embodiments, the third measurement zone 308 can include the same (in type and/or number) discrete graphene varactors that are within the first measurement zone 304. In some embodiments, the third measurement zone 308 can include only a subset of the discrete graphene varactors that are within the first measurement zone 304. In some embodiments, the third measurement zone 308 can include discrete graphene varactors that are different than those of the first measurement zone 304 and the second measurement zone 306. Aspects of the third measurement zone are described in greater detail below.

The first measurement zone, the second measurement zone, and the third measurement zone can be the same size or can be of different sizes. The chemical sensor element 300 can also include a component 310 to store reference data. The component 310 to store reference data can be an electronic data storage device, an optical data storage device, a printed data storage device (such as a printed code), or the like. The reference data can include, but is not limited to, data regarding the third measurement zone (described in greater detail below).

In some embodiments, chemical sensor elements embodied herein can include electrical contacts (not shown) that can be used to provide power to components on the chemical sensor element 300 and/or can be used to read data regarding the measurement zones and/or data from the stored in component 310. However, in other embodiments there are no external electrical contacts on the chemical sensor element 300. Various additional components of the chemical sensor elements herein are described in U.S. App. No. 62/898,155, the content of which is herein incorporated by reference.

It will be appreciated that many different circuit designs can be used to gather data and/or signals from chemical sensor elements herein including both direct-contact circuit designs as well as passive wireless sensing circuit designs. Some exemplary measurement circuits are described in U.S. Publ. Appl. No. 2019/0025237, the content of which is herein incorporated by reference.

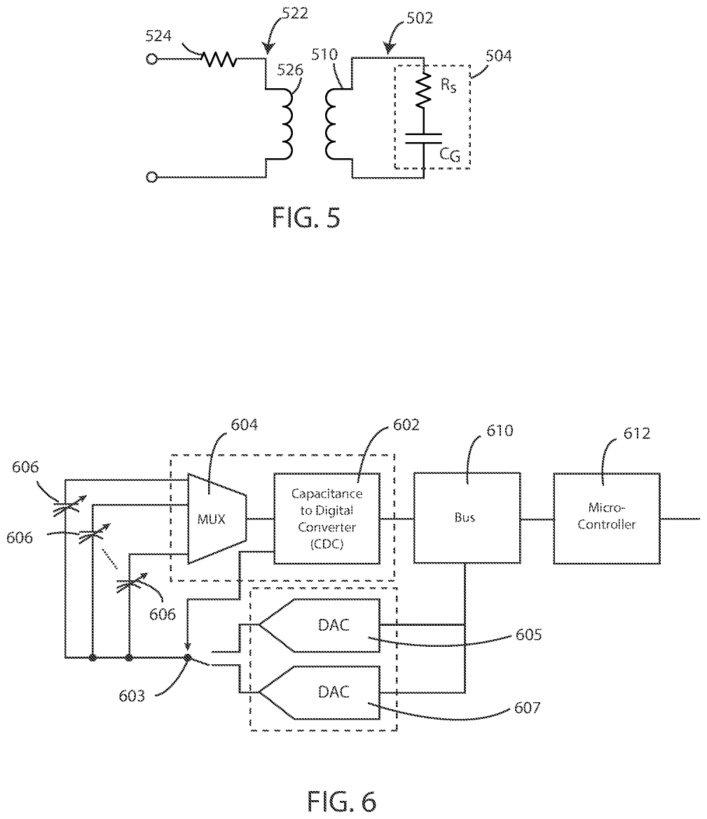

It will be appreciated that the chemical sensor elements embodied herein can include those that are compatible with passive wireless sensing. A schematic diagram of a passive sensor circuit 502 and a portion of a reading circuit 522 is shown in FIG. 5 and discussed in more detail below. In the passive wireless sensing arrangement, the graphene varactor(s) can be integrated with an inductor such that one terminal of the graphene varactor contacts one end of the inductor, and a second terminal of the graphene varactor contacts a second terminal of the inductor. In some embodiments, the inductor can be located on the same substrate as the graphene varactor, while in other embodiments, the inductor could be located in an off-chip location.

Referring now to FIG. 4, a schematic diagram of a portion of a measurement zone 400 is shown in accordance with various embodiments herein. A plurality of discrete graphene varactors 402 can be disposed within the measurement zone 400 in an array. In some embodiments, a chemical sensor element can include a plurality of graphene varactors configured in an array within a measurement zone. In some embodiments, the plurality of graphene varactors can be identical, while in other embodiments the plurality of graphene varactors can be different from one another.

In some embodiments, the discrete graphene varactors can be heterogeneous in that they are all different from one another in terms of their binding behavior specificity with regard to a particular analyte. In some embodiments, some discrete graphene varactors can be duplicated for validation purposes, but are otherwise heterogeneous from other discrete graphene varactors. Yet in other embodiments, the discrete graphene varactors can be homogeneous in that they are the same in terms of their binding behavior specificity with regard to a particular analyte. While the discrete graphene varactors 402 of FIG. 4 are shown as boxes organized into a grid, it will be appreciated that the discrete graphene varactors can take on many different shapes (including, but not limited to, various polygons, circles, ovals, irregular shapes, and the like) and, in turn, the groups of discrete graphene varactors can be arranged into many different patterns (including, but not limited to, star patterns, zig-zag patterns, radial patterns, symbolic patterns, and the like).

In some embodiments, the order of specific discrete graphene varactors 402 across the length 412 and width 414 of the measurement zone can be substantially random. In other embodiments, the order can be specific. For example, in some embodiments, a measurement zone can be ordered so that the specific discrete graphene varactors 402 for analytes having a lower molecular weight are located farther away from the incoming gas flow relative to specific discrete graphene varactors 402 for analytes having a higher molecular weight which are located closer to the incoming gas flow. As such, chromatographic effects which may serve to provide separation between chemical compounds of different molecular weight can be taken advantage of to provide for optimal binding of chemical compounds to corresponding discrete graphene varactors.

The number of discrete graphene varactors within a particular measurement zone can be from about 1 to about 100,000. In some embodiments, the number of discrete graphene varactors can be from about 1 to about 10,000. In some embodiments, the number of discrete graphene varactors can be from about 1 to about 1,000. In some embodiments, the number of discrete graphene varactors can be from about 2 to about 500. In some embodiments, the number of discrete graphene varactors can be from about 10 to about 500. In some embodiments, the number of discrete graphene varactors can be from about 50 to about 500. In some embodiments, the number of discrete graphene varactors can be from about 1 to about 250. In some embodiments, the number of discrete graphene varactors can be from about 1 to about 50.

Each of the discrete graphene varactors suitable for use herein can include at least a portion of one or more electrical circuits. By way of example, in some embodiments, each of the discrete graphene varactors can include one or more passive electrical circuits. In some embodiments, the graphene varactors can be included such that they are integrated directly on an electronic circuit. In some embodiments, the graphene varactors can be included such that they are wafer bonded to the circuit. In some embodiments, the graphene varactors can include integrated readout electronics, such as a readout integrated circuit (ROIC). The electrical properties of the electrical circuit, including resistance or capacitance, can change upon binding, such as specific and/or non-specific binding, with a component from a gas sample.

Referring now to FIG. 5, a schematic diagram of a passive sensor circuit 502 and a portion of a reading circuit 522 is shown in accordance with various aspects herein. In some embodiments, the passive sensor circuit 502 can include a metal-oxide—graphene varactor 504 (wherein RS represents the series resistance and CG represents the varactor capacitor) coupled to an inductor 510. Graphene varactors can be prepared in various ways and with various geometries. By way of example, in some aspects, a gate electrode can be recessed into an insulator layer as shown as gate electrode 104 in FIG. 1. A gate electrode can be formed by etching a depression into the insulator layer and then depositing an electrically conductive material in the depression to form the gate electrode. A dielectric layer can be formed on a surface of the insulator layer and the gate electrode. In some examples, the dielectric layer can be formed of a metal oxide such as, aluminum oxide, hafnium dioxide, zirconium dioxide, silicon dioxide, or of another material such as hafnium silicate or zirconium silicate. A surface-modified graphene layer can be disposed on the dielectric layer. Contact electrodes can also be disposed on a surface of the surface-modified graphene layer, also shown in FIG. 1 as contact electrode 110.

Further aspects of exemplary graphene varactor construction can be found in U.S. Pat. No. 9,513,244, the content of which is herein incorporated by reference in its entirety.

In various embodiments, the functionalized graphene layer (e.g., functionalized to include analyte binding receptors), which is part of the graphene varactor and thus part of a sensor circuit, such as a passive sensor circuit, is exposed to the gas sample flowing over the surface of the measurement zone. The passive sensor circuit 502 can also include an inductor 510. In some embodiments, only a single varactor is included with each passive sensor circuit 502. In other embodiments, multiple varactors are included, such as in parallel, with each passive sensor circuit 502.

In the passive sensor circuit 502, the capacitance of the electrical circuit changes upon binding of an analyte in the gas sample and the graphene varactor. The passive sensor circuit 502 can function as an LRC resonator circuit, wherein the resonant frequency of the LRC resonator circuit changes upon binding with a component from a gas sample.

The reading circuit 522 can be used to detect the electrical properties of the passive sensor circuit 502. By way of example, the reading circuit 522 can be used to detect the resonant frequency of the LRC resonator circuit and/or changes in the same. In some embodiments, the reading circuit 522 can include a reading coil having a resistance 524 and an inductance 526. When the sensor-side LRC circuit is at its resonant frequency, a plot of the phase of the impedance of the reading circuit versus the frequency has a minimum (or phase dip frequency). Sensing can occur when the varactor capacitance varies in response to binding of analytes, which changes the resonant frequency, and/or the value of the phase dip frequency.

The capacitance of the graphene varactors can be measured by delivering an excitation current at a particular voltage and/or over a range of voltages. Measuring the capacitance provides data that reflects the binding status of analytes to the graphene varactor(s). Various measurement circuitry can be used to measure the capacitance of the graphene varactor(s).

Referring now to FIG. 6, a schematic diagram is shown of circuitry to measure the capacitance of a plurality of discrete graphene varactors in accordance with various embodiments herein. The circuitry can include a capacitance to digital converter (CDC) 602 in electrical communication with a multiplexor 604. The multiplexor 604 can provide selective electrical communication with a plurality of graphene varactors 606. The connection to the other side of the graphene varactors 606 can be controlled by a switch 603 (as controlled by the CDC) and can provide selective electrical communication with a first digital to analog converter (DAC) 605 and a second digital to analog converter (DAC) 607. The other side of the DACs 605, 607 can be connected to a bus device 610, or in some cases, the CDC 602. The circuitry can further include a microcontroller 612, which will be discussed in more detail below.

In this case, the excitation signal from the CDC controls the switch between the output voltages of the two programmable Digital to Analog Converters (DACs). The programmed voltage difference between the DACs determines the excitation amplitude, providing an additional programmable scale factor to the measurement and allowing measurement of a wider range of capacitances than specified by the CDC. The bias voltage at which the capacitance is measured is equal to the difference between the bias voltage at the CDC input (via the multiplexor, usually equal to VCC/2, where VCC is the supply voltage) and the average voltage of the excitation signal, which is programmable. In some embodiments, buffer amplifiers and/or bypass capacitance can be used at the DAC outputs to maintain stable voltages during switching. Many different ranges of DC bias voltages can be used. In some embodiments, the range of DC bias voltages can be from −3 V to 3 V, or from −1 V to 1 V, or from −0.5 V to 0.5 V.

Many different aspects can be calculated based on the capacitance data. For example, aspects that can be calculated include maximum slope of capacitance to voltage, change in maximum slope of capacitance to voltage over a baseline value, minimum slope of capacitance to voltage, change in minimum slope of capacitance to voltage over a baseline value, minimum capacitance, change in minimum capacitance over a baseline value, voltage at minimum capacitance (Dirac point), change in voltage at minimum capacitance, maximum capacitance, change in maximum capacitance, ratio of maximum capacitance to minimum capacitance, response time constants, and ratios of any of the foregoing between different discrete graphene varactors and particularly between different discrete graphene varactors having specificity for different analytes.

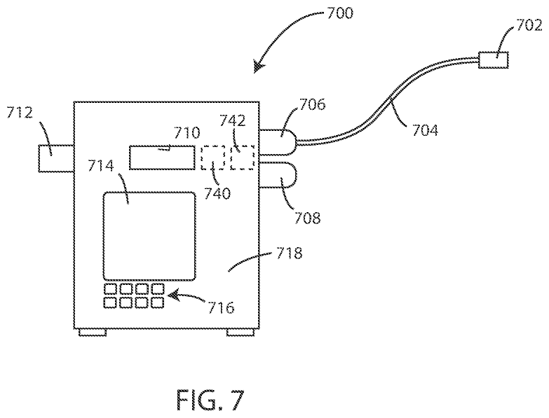

Referring now to FIG. 7, a schematic view of a system 700 for sensing gaseous analytes in accordance with various embodiments herein is shown. The system 700 can include a housing 718. The system 700 can include a mouthpiece 702 into which a subject to be evaluated can blow a breath sample. The gaseous breath sample can pass through an inflow conduit 704 and pass through an evaluation sample (patient sample) input port 706. The system 700 can also include a control sample (environment) input port 708. The system 700 can also include a sensor element chamber 710, into which disposable sensor elements can be placed. When placed into a sensor element chamber, the disposable sensor elements and portions thereof can define one or more gas flow paths. The system 700 can also include a display screen 714 and a user input device 716, such as a keyboard. The system can also include a gas outflow port 712. The system 700 can also include flow sensors in fluid communication with the gas flow associated with one or more of the evaluation sample input port 706 and control sample input port 708. It will be appreciated that many different types of flow sensors can be used. In some embodiments, a hot-wire anemometer can be used to measure the flow of air. In some embodiments, the system can include a CO₂ sensor in fluid communication with the gas flow associated with one or more of the evaluation sample input port 706 and control sample input port 708. Additional methods for data analysis and the kinetics of response for the chemical sensors herein can be found in U.S. application Ser. No. 16/712,255, the content of which is herein incorporated by reference.

In various embodiments, the system 700 can also include other functional components. By way of example, the system 700 can include a humidity control module 740 and/or a temperature control module 742. The humidity control module can be in fluid communication with the gas flow associated with one or more of the evaluation sample input port 706 and control sample input port 708 in order to adjust the humidity of one or both gas flow streams in order to make the relative humidity of the two streams substantially the same in order to prevent an adverse impact on the readings obtained by the system. The temperature control module can be in fluid communication with the gas flow associated with one or more of the evaluation sample input port 706 and control sample input port 708 in order to adjust the temperature of one or both gas flow streams in order to make the temperature of the two streams substantially the same in order to prevent an adverse impact on the readings obtained by the system. By way of example, the air flowing into the control sample input port can be brought up to 37 degrees Celsius or higher in order to match or exceed the temperature of air coming from a patient. The humidity control module and the temperature control module can be upstream from the input ports, within the input ports, or downstream from the input ports in the housing 718 of the system 700. In some embodiments, the humidity control module 740 and the temperature control module 742 can be integrated.

In some embodiments (not shown), the control sample input port 708 of system 700 can also be connected to a mouthpiece 702. In some embodiments, the mouthpiece 702 can include a switching airflow valve such that when the patient is drawing in breath, air flows from the control sample input port 708 to the mouthpiece, and the system is configured so that this causes ambient air to flow across the appropriate control measurement zone (such as the second measurement zone). Then when the patient exhales, the switching airflow valve can switch so that a breath sample from the patient flows from the mouthpiece 702 through the inflow conduit 704 and into the evaluation sample input port 706 and across the appropriate sample (patient sample) measurement zone (such as the first measurement zone) on the disposable sensor element.

In an embodiment, a method of making a chemical sensor element is included. The method can include depositing one or more measurement zones onto a substrate. The method can further include depositing a plurality of discrete graphene varactors within the measurement zones on the substrate. The method can include generating one or more discrete graphene varactors by modifying a surface of a graphene layer one or more nanoparticles as described herein to form a non-covalent modification layer on an outer surface of the graphene layer through non-covalent interactions. The method can include quantifying the extent of surface coverage of the non-covalent modification layer using contact angle goniometry, Raman spectroscopy, or X-Ray photoelectron spectroscopy. The method can further include depositing a component to store reference data onto the substrate. In some embodiments, the measurement zones can all be placed on the same side of the substrate. In other embodiments, the measurement zones can be placed onto different sides of the substrate.

In an embodiment, a method of assaying one or more gas samples is included. The method can include inserting a chemical sensor element into a sensing machine. The chemical sensor element can include a substrate and a first measurement zone comprising a plurality of discrete graphene varactors. The first measurement zone can define a portion of a first gas flow path. The chemical sensor element can further include a second measurement zone separate from the first measurement zone. The second measurement zone can also include a plurality of discrete graphene varactors. The second measurement zone can be disposed outside of the first gas flow path.

The method can further include prompting a subject to blow air into the sensing machine to follow the first gas flow path. In some embodiments, the CO₂ content of the air from the subject is monitored and sampling with the disposable sensor element is conducted during the plateau of CO₂ content, as it is believed that the air originating from the alveoli of the patient has the richest content of chemical compounds for analysis, such as volatile organic compounds. In some embodiments, the method can include monitoring the total mass flow of the breath sample and the control (or environmental) air sample using flow sensors. The method can further include interrogating the discrete graphene varactors to determine their analyte binding status. The method can further include discarding the disposable sensor element upon completion of sampling.

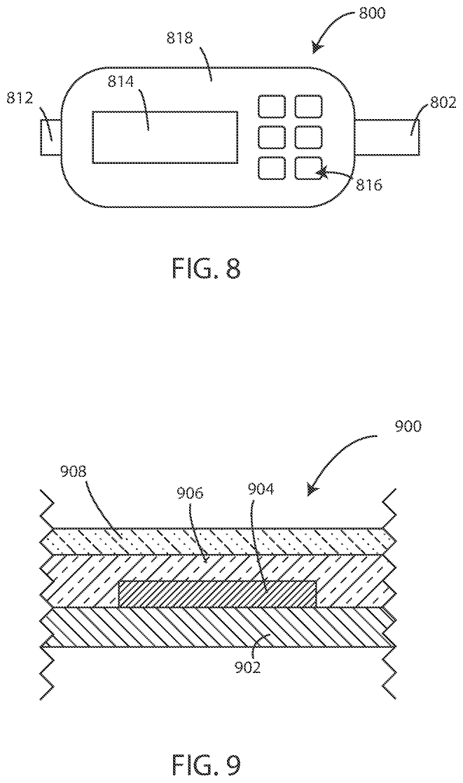

Referring now to FIG. 8, a schematic view of a system 800 for sensing gaseous analytes in accordance with various embodiments herein is shown. In this embodiment, the system is in a hand-held format. The system 800 can include a housing 818. The system 800 can include a mouthpiece 802 into which a subject to be evaluated can blow a breath sample. The system 800 can also include a display screen 814 and a user input device 816, such as a keyboard. The system can also include a gas outflow port 812. The system can also include various other components such as those described with reference to FIG. 7 above.

In some embodiments, one of the measurement zones can be configured to indicate changes (or drift) in the chemical sensor element that could occur as a result of aging and exposure to varying conditions (such as heat exposure, light exposure, molecular oxygen exposure, humidity exposure, etc.) during storage and handling prior to use. In some embodiments, the third measurement zone can be configured for this purpose.

Referring now to FIG. 9, a schematic cross-sectional view is shown of a portion of a chemical sensor element 900 in accordance with various embodiments herein. The chemical sensor element 900 can include a substrate 902 and a discrete graphene varactor 904 disposed thereon that is part of a measurement zone. Optionally, in some embodiments the discrete graphene varactor 904 can be encapsulated by an inert material 906, such as nitrogen gas, or an inert liquid or solid. In this manner, the discrete graphene varactor 904 for the third measurement zone can be shielded from contact with gas samples and can therefore be used as a control or reference to specifically control for sensor drift which may occur between the time of manufacturing and the time of use of the disposable sensor element. In some embodiments, such as in the case of the use of an inert gas or liquid, the discrete binding detector can also include a barrier layer 908, which can be a layer of a polymeric material, a foil, or the like. In some cases, the barrier layer 908 can be removed just prior to use.

In an embodiment, a method for detecting one or more analytes is included. The method can include collecting a gaseous sample from a patient. In some embodiments the gaseous sample can include exhaled breath. In other embodiments, the gaseous sample can include breath removed from the lungs of a patient via a catheter or other similar extraction device. In some embodiments, the extraction device can include an endoscope, a bronchoscope, or tracheoscope. The method can also include contacting a graphene varactor with the gaseous sample, where the graphene varactor includes a graphene layer and a non-covalent modification layer disposed on an outer surface of the graphene layer through non-covalent interactions. In some embodiments, the method can include measuring a differential response in a capacitance of the graphene reactor due to the binding of one or more analytes present in the gaseous sample, which in turn can be used to identify disease states. In some embodiments, the method can include a non-covalent modification layer selected from at least one nanoparticle as described herein, or derivatives thereof.

### Graphene Varactors

The graphene varactors described herein can be used to sense one or more analytes in a gaseous sample, such as, for example, the breath of a patient. Graphene varactors embodied herein can exhibit a high sensitivity for volatile organic compounds (VOCs) found in gaseous samples at or near parts-per-million (ppm) or parts-per-billion (ppb) levels. The adsorption of VOCs onto the surface of graphene varactors can change the resistance, capacitance, or quantum capacitance of such devices, and can be used to detect the VOCs and/or patterns of binding by the same that, in turn, can be used to identify disease states such as cancer, cardiac diseases, infections, multiple sclerosis, Alzheimer's disease, Parkinson's disease, and the like. The graphene varactors can be used to detect individual analytes in gas mixtures, as well as patterns of responses in highly complex mixtures. In some embodiments, one or more graphene varactors can be included to detect the same analyte in a gaseous sample. In some embodiments, one or more graphene varactors can be included to detect different analytes in a gaseous sample. In some embodiments, one or more graphene varactors can be included to detect a multitude of analytes in a gaseous sample. The graphene varactors described herein can include those in which a single graphene layer has been surface-modified through non-covalent interactions with one or more nanoparticles.

### X-Ray Photoelectron Spectroscopy

X-ray photoelectron spectroscopy (XPS) is a highly sensitive spectroscopic technique that can quantitatively measure the elemental composition of a surface of a material. The process of XPS involves irradiation of a surface with X-rays under a vacuum, while measuring the kinetic energy and electron release within the top 0 to 10 nm of a material. Without wishing to be bound by any particular theory, it is believed that XPS can be used to confirm the presence of a modification layer disposed on the surface of graphene.

The surface concentrations of the types of atoms that the modification layer, graphene, and the underlying substrate consist of (as determined from XPS) depends on the monolayer molecules on the graphene. For example, the surface concentrations of carbon, oxygen, and copper (i.e., C %, O %, and Cu %, as determined from XPS) for graphene (grown on a copper substrate) modified with any given nanoparticle depend on the concentration of that nanoparticle in solution or suspension.

### Contact Angle Goniometry

Contact angle goniometry can be used to determine the wettability of a solid surface by a liquid. Wettability, or wetting, can result from the intermolecular forces at the contact area between a liquid and a solid surface. The degree of wetting can be described by the value of the contact angle Φ formed between the area of contact between the liquid and the solid surface and a line tangent to the liquid-vapor interface. When a surface of a solid is hydrophilic and water is used as the test liquid, (i.e., a high degree of wettability), the value for Φ can fall within a range of 0 to 90 degrees. When a surface of a solid is moderately hydrophilic to hydrophobic, (i.e., a medium degree of wettability), the value for Φ for water as the test liquid can fall within a range of 85 to 105 degrees. When the surface of a solid is highly hydrophobic, (i.e., a low degree of wettability), the value for Φ with water as the test liquid can fall within a range of 90 to 180 degrees. Thus, a change in contact angle can be reflective of a change in the surface chemistry of a substrate.

Graphene surfaces and modifications made to graphene surfaces can be characterized using contact angle goniometry. Contact angle goniometry can provide quantitative information regarding the degree of modification of the graphene surface. Contact angle measurements are highly sensitive to the groups present on sample surfaces and can be used to determine the formation and extent of surface coverage of self-assembled monolayers. A change in the contact angle from a bare graphene surface as compared to one that has been modified with a functional layer, can be used to confirm the formation of the functional layer on the surface of the graphene.

The types of solvents suitable for use in determining contact angle measurements, also called wetting solutions, are those that maximize the difference between the contact angle of the solution on bare graphene and the contact angle on the modified graphene, thereby improving data accuracy for measurements of binding isotherms. In some embodiments, the wetting solutions can include, but are not limited to, deionized (DI) water, NaOH aqueous solution, borate buffer (pH 9.0), other pH buffers, CF₃CH₂OH, and the like. In some embodiments, the wetting solutions are polar. In some embodiments, the wetting solutions are non-polar.

### Characterization of Surface Modification by Nanoparticles Using Capacitance

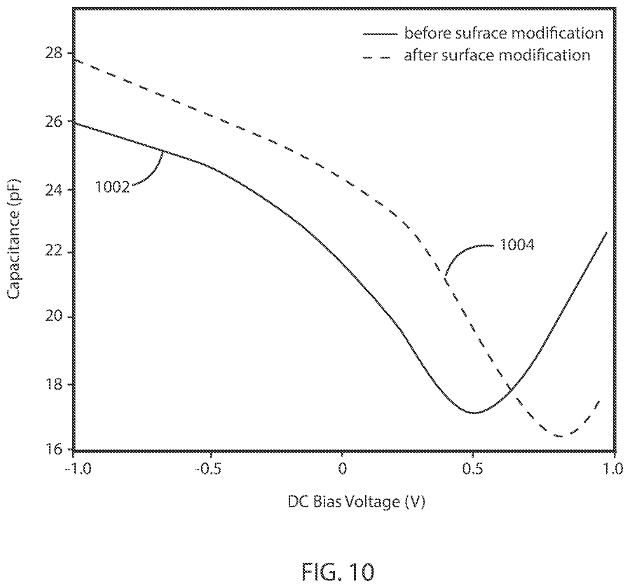

When a graphene surface is modified by one or more nanoparticles, as described herein, the response signal of the graphene can change when compared to the baseline response signal in the absence of nanoparticle. Referring now to FIG. 10, response signals for an individual graphene varactor before and after surface modification are shown on a graph of capacitance versus DC bias voltage in accordance with various embodiments herein. The response signal for the graphene varactor before exposure to one or more nanoparticles shown in plot 1002. The response signal for the same graphene varactor after modification with one or more nanoparticles is shown in plot 1004. Response signals, such the capacitance versus voltage curve shown in FIG. 10, can be established by measuring capacitance over a range of DC bias voltages (an example of an excitation cycle), both before and after surface modification with one or more nanoparticles.

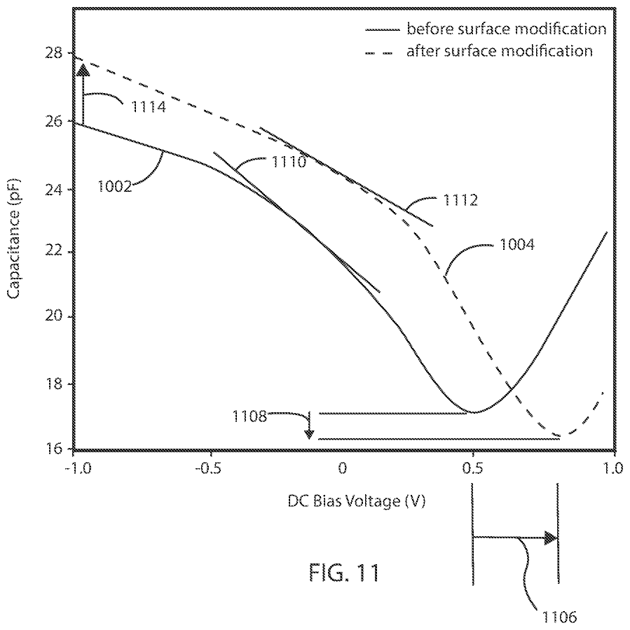

Several different parameters of the graphene varactor response signal can change from a baseline value to a higher or a lower value, and the shape of the response signal can change in response to surface modification with nanoparticles. Referring now to FIG. 11, the same response signals for an individual graphene varactor before and after exposure to a gaseous mixture are shown that were shown in FIG. 10, but with various annotations provided to highlight the change in the different parameters of the graphene varactor response signal that can be analyzed to characterize the modification of the surface of graphene by nanoparticles. By way of example, these different parameters can include, but are not to be limited to, a shift in the Dirac point (i.e., the voltage when the capacitance of a graphene varactor is at a minimum), a change in the minimum capacitance of the graphene varactor, a change in the slope of the response signal, or the change in the maximum capacitance of the graphene varactor, change in capacitance at a particular bias voltage, or the like (other examples of parameters are described below).

In FIG. 11, the response signal for the graphene varactor before surface modification with nanoparticles is shown as plot 1002, while the response signal for the same graphene varactor after surface modification with nanoparticles is shown as plot 1004. The shift in the Dirac point is indicated as arrow 1106. The change in the minimum capacitance of the graphene varactor is indicated as arrow 1108. The change in the slope of the response signal can be obtained by comparison of the slope 1110 of plot 1002 for the graphene varactor before surface modification with nanoparticles with the slope 1112 of plot 1004 for the graphene varactor after surface modification with nanoparticles. The change in the maximum capacitance of the graphene varactor is indicated as arrow 1114.

In some embodiments, a ratio of the maximum capacitance to minimum capacitance can be used to characterize the surface modification with nanoparticles. In some embodiments, a ratio of the maximum capacitance to the shift in the Dirac point can be used to characterize the surface modification with nanoparticles. In other embodiments, a ratio of the minimum capacitance to the shift in the slope of the response signal can be used to characterize the surface modification with nanoparticles. In some embodiments, a ratio of any of the parameters including a shift in the Dirac point, a change in the minimum capacitance, a change in the slope of the response signal, or the change in the maximum capacitance can be used to characterize the surface modification with nanoparticles.

### Nanoparticles and Modified Nanoparticles

As discussed herein, nanoparticles can include one or more of gold (Au), platinum (Pt), silver (Ag), palladium (Pd), iron III oxide (Fe₂O₃), iron II, III oxide (Fe₃O₄), zinc oxide (ZnO), palladium II oxide (PdO), tin dioxide (SnO₂), titanium (Ti), titanium dioxide (TiO₂), silicon dioxide (SiO₂), cobalt diiron tetraoxide (CoFe₂O₄), indium trioxide (In₂O₃), vanadium pentoxide (V₂O₅), platinum oxide (PtO₂), copper oxide (CuO), cadmium oxide (CdO), chromium niobate (CrNbO₄), CoNb₂O₆, molybdenum disulfide (MoS₂), tungsten oxide (WO), tungsten dioxide (WO₂), tungsten trioxide (WO₃), neodymium oxide (Nd₂O₃), boron nitride (BN), CeFeO₄H, manganese oxide (Mn₃O₄), any form of iron oxyhydroxide (FeOOH, which includes goethite, akageneite, lepidocrocite, and feroxyhyte, or any other form of FeOOH), or any combinations or derivatives thereof.

In various embodiments, the nanoparticles herein can include modifications such as with alkylthio, alkenylthio, alkynylthio, heteroalkylthio, heteroalkenylthio, heteroalkynylthio, haloalkylthio, haloalkenylthio, haloalkynylthio, halogenated heteroalkylthio, halogenated heteroalkenylthio, halogenated heteroalkynylthio, arylthio, substituted arylthio, heteroarylthio, or substituted heteroarylthio groups as described herein.

Modification of nanoparticles with the groups described herein can include various synthetic reaction schemes, including without limitation the formation of self-assembled monolayers, by ligand-exchange reactions, or by various reduction methods. As described in U.S. Application No. US 2009/0104435A1, which is incorporated herein by reference, various ligand exchange reactions can be utilized to modify gold nanoparticles with thio groups. The reduction methods suitable for use herein can include or be based on those as reported by Turkevich et al., (J. Turkevich, P. C. Stevenson and J. Hillier, Discuss. Faraday Soc., 1951, 11, 55) and Brust and Schiffrin (M. Brust, M. Walker, D. Bethell, D. J. Schiffrin and R. Whyman, J. Chem. Soc., Chem. Commun., 1994, 801; M. Brust, D. Bethell, D. J. Schiffrin and C. Kiely, Adv. Mater., 1995, 7, 795), which are incorporated herein by reference.

In various embodiments, the nanoparticles herein can be modified by using a self-assembled monolayer reaction. A solution of any of the thiol compounds used to create the thio modifications on the surface of nanoparticles herein can be created in an alcohol or another suitable solvent or solvent mixture. In various embodiments the alcohol can include ethanol. The concentration of thiol compounds used the self-assembly of thio compounds on the surface of the nanoparticles can include at least 0.5 mM in ethanol. In various embodiments, the concentration of thiol compound can include at least 1.0 mM in ethanol. In other embodiments, the concentration of thiol compound can be up to a maximum solubility of the thiol compound prior to saturation of the solution with the compound. Gold nanoparticles can be added to the thiol compound in ethanol an allowed to incubate in solution for about three hours. In various embodiments the solution of thiol compound and gold nanoparticles can be agitated during incubation. The incubation can take place at from about 20° C. to about 30° C. In various embodiments the incubation can take place at room temperature or about 25° C. The gold nanoparticles can be removed and rinsed in solvent until use.

It will be appreciated that metal nanoparticles can be modified with thio compounds in various ways. In some embodiments, gold nanoparticles can be modified with thio compounds in the presence of a solvent and a reducing agent. By way of example, the thio-modification of gold nanoparticles has been reported by Brust and Schiffrin using a two-phase reduction of chloroauric acid (HAuCl₄) with reducing agent in the presence of an alkyl thiol compound. Various reducing agents are suitable for use herein, including, but not to be limited to sodium borohydrate, trisodium citrate, and lithium triethylborohydride. In yet other embodiments, the gold nanoparticles can be reduced using amine reducing agents. In some embodiments, the reduction reactions can take place in toluene, tetrahydrofuran (THF), and the like.

### Methods

Many different methods are contemplated herein, including, but not limited to, methods of making, methods of using, and the like. Aspects of system/device operation described elsewhere herein can be performed as operations of one or more methods in accordance with various embodiments herein.

In an embodiment, a method of modifying a surface of graphene, the method is included, the method contacting a graphene layer with a solution or suspension can include one or more nanoparticles can include metals, metal oxides, or derivatives thereof, forming at least one non-covalent modification layer of the one or more nanoparticles disposed on an outer surface of a graphene layer, wherein the at least one non-covalent modification layer comprises one or more nanoparticles selected from the group can include one or more metals, metal oxides, or derivatives thereof, and quantifying an extent of surface coverage of the at least one non-covalent modification layer using contact angle goniometry, Raman spectroscopy, scanning electron microscopy (SEM), transmission electron microscopy (TEM), atomic force microscopy (AFM), scanning tunneling microscopy (STM), or X-Ray photoelectron spectroscopy.

In an embodiment, the method can include contacting a graphene layer with a solution or a suspension by immersing the graphene layer into the suspension. In other embodiments, the method can include contacting a graphene layer with a solution or a suspension by spray coating, drop coating, or spin coating the graphene layer with the solution or the suspension.

In an embodiment, a method for detecting an analyte is included, the method collecting a gaseous sample, contacting the gaseous sample with one or more graphene varactors, each of the one or more graphene varactors is included, the method a graphene layer, at least one non-covalent modification layer disposed on an outer surface of the graphene layer, and wherein the at least one non-covalent modification layer comprises one or more nanoparticles selected from the group can include metals, metal oxides, or derivatives thereof.

In an embodiment, the gaseous sample can include a patient breath sample or an environmental gas sample.

In an embodiment, the method can further include measuring a differential response in an electrical property of the one or more graphene varactors due to binding of one or more analytes present in the gaseous sample.

In an embodiment, the electrical property selected from the group including of capacitance or resistance.

In an embodiment, the nanoparticles can include metals or metal oxides selected from the group including gold (Au), platinum (Pt), silver (Ag), palladium (Pd), iron III oxide (Fe₂O₃), iron II, III oxide (Fe₃O₄), zinc oxide (ZnO), palladium II oxide (PdO), tin dioxide (SnO₂), titanium (Ti), titanium dioxide (TiO₂), silicon dioxide (SiO₂), cobalt diiron tetraoxide (CoFe₂O₄), indium trioxide (In₂O₃), vanadium pentoxide (V₂O₅), platinum oxide (PtO₂), copper oxide (CuO), cadmium oxide (CdO), chromium niobate (CrNbO₄), CoNb₂O₆, molybdenum disulfide (MoS₂), tungsten oxide (WO), tungsten dioxide (WO₂), tungsten trioxide (WO₃), neodymium oxide (Nd₂O₃), boron nitride (BN), CeFeO₄H, manganese oxide (Mn₃O₄), any form of iron oxyhydroxide (FeOOH, which includes goethite, akageneite, lepidocrocite, and feroxyhyte, or any other form of FeOOH), and any combinations, or derivatives thereof.

In an embodiment of the method, the nanoparticles are further modified with groups can include alkylthio, alkenylthio, alkynylthio, heteroalkylthio, heteroalkenylthio, heteroalkynylthio, haloalkylthio, haloalkenylthio, haloalkynylthio, halogenated heteroalkylthio, halogenated heteroalkenylthio, halogenated heteroalkynylthio, arylthio, substituted arylthio, heteroarylthio, or substituted heteroarylthio.

Aspects may be better understood with reference to the following examples. These examples are intended to be representative of specific embodiments, but are not intended as limiting the overall scope of embodiments herein.

### EXAMPLES

### Experimental Materials

Gold nanoparticles (Au NPs) having an average diameter of 5 nm were suspended in a 0.1 M phosphate buffered saline (PBS) solution at a concentration of 5.5×10¹³ particles/mL. 1-octanethiol functionalized gold nanoparticles (C₈—S—Au NPs) having an average diameter of 2-4 nm were suspended in a toluene solution at a concentration of 2% w/v. The gold nanoparticles and 1-octanethiol functionalized gold nanoparticles were purchased from Aldrich (St. Louis, Mo.). Monolayer graphene on Cu foil grown by chemical vapor deposition was purchased from Graphenea (Donostia, Spain).

### Example 1: Graphene Surface Modification With Nanoparticles

The suspension of Au NPs was diluted to 4.1*10¹³ particles/mL using ethanol in order to wet the graphene surface. Graphene substrates were immersed overnight into the Au NPs suspension and then washed 3 times with small portions of ethanol to remove excess Au NPs suspension. Graphene substrates were immersed overnight into the C₈—S—Au NPs suspension and then washed 3 times with small portions of toluene to remove excess C₈—S—Au NPs suspension.

### Example 2: Surface Characterization Using XPS

X-ray photoelectron spectroscopy (XPS) spectra of bare graphene and nanoparticle functionalized graphene were collected on a VersaProbe III Scanning XPS Microprobe (PHI 5000, 5 Physical Electronics, Chanhassen, Minn.). The results for the elemental surface composition of functionalized graphene are shown in Table 1.

TABLE 1
Elemental surface composition of nanoparticle functionalized
graphene as determined by XPS

| | Before | | | After | | |
|---|---|---|---|---|---|---|
| Nanoparticles | functionalization | | | functionalization | | |
| Au NPs | C | % | 47.7 ± 0.4 | C | % | 51.3 ± 1.5 |
| | Cu | % | 45.8 ± 1.0 | Cu | % | 17.7 ± 0.8 |
| | O | % | 6.2 ± 0.7 | O | % | 26.4 ± 0.4 |
| | Au | % | n/a | Au | % | 4.5 ± 0.4 |
| C₈—S—Au NPs | C | % | 47.7 ± 0.4 | C | % | 52.7 ± 0.6 |
| | Cu | % | 45.8 ± 1.0 | Cu | % | 38.7 ± 1.4 |
| | O | % | 6.2 ± 0.7 | O | % | 6.9 ± 0.7 |
| | Au | % | n/a | Au | % | 1.6 ± 0.4 |

### Example 3: Graphene Varactor Surface Characterization

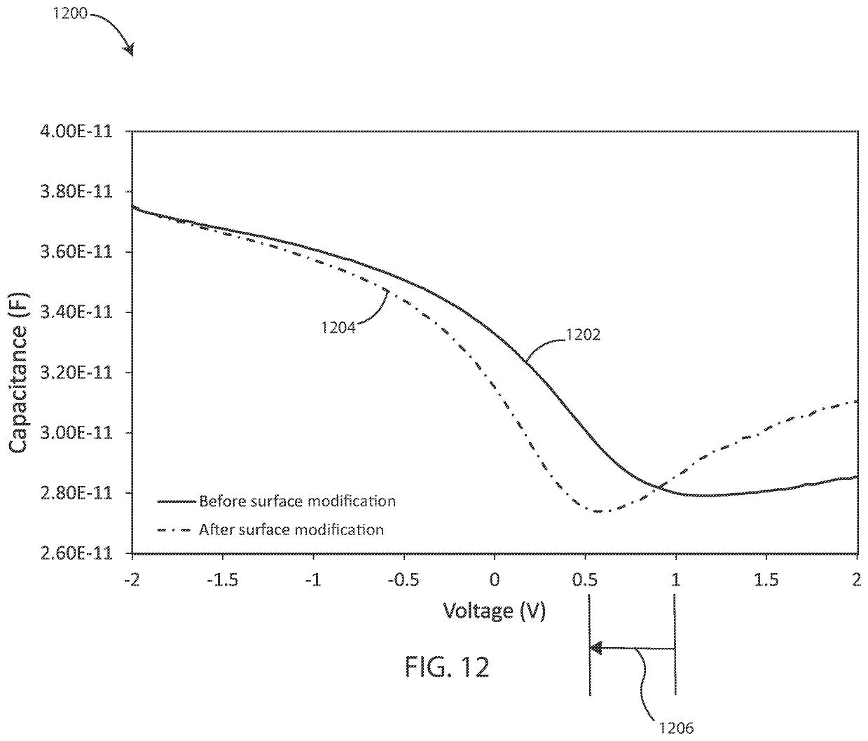

To determine the Dirac point of graphene-based varactors modified with gold nanoparticles (Au NPs) and 1-octanethiol gold nanoparticles (C₈—S—Au NPs), capacitance-voltage (C-V) measurements were performed. As shown in FIG. 12, the response signal for a graphene varactor before functionalization and after functionalization with gold nanoparticles (Au NPs) is shown in graph 1200. The response signal for a graphene varactor before exposure gold nanoparticles (Au NPs) is shown in plot 1202. The response signal for the same graphene varactor after modification with gold nanoparticles (Au NPs) is shown in plot 1204. As shown in the graph, the forward Dirac point of the graphene varactor shifts to the left from 1.0 V (before surface modification) to 0.5 V (after surface modification with Au NPs), to give a shift of approximately 0.5 V. The shift in the Dirac point for graphene modification with gold nanoparticles is indicated as arrow 1206.

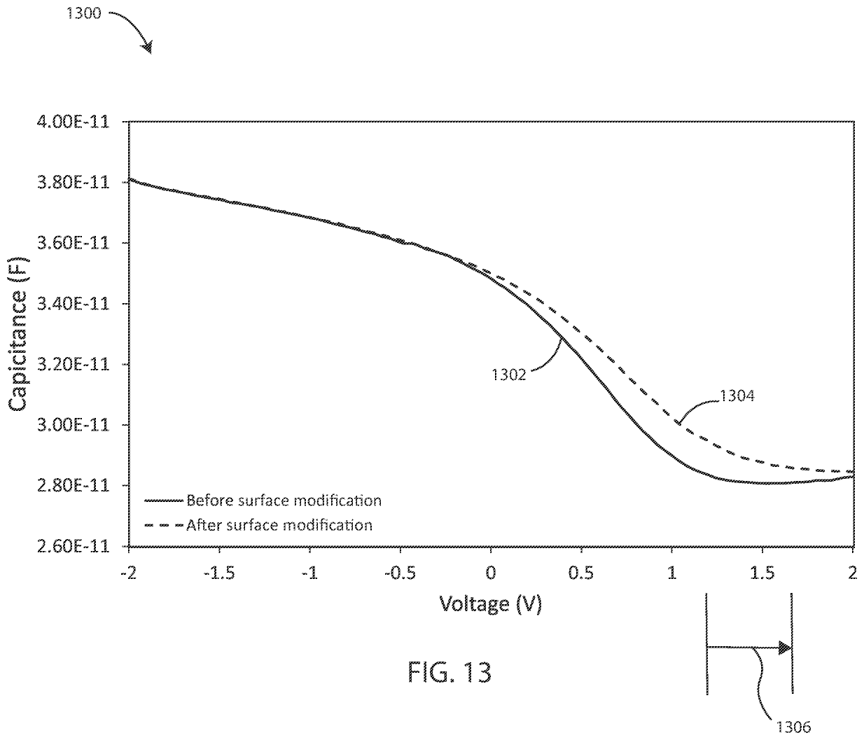

As shown in FIG. 13, the response signal for a graphene varactor before functionalization and after functionalization with 1-octanethiol gold nanoparticles (C₈—S—Au NPs) is shown in graph 1300. The response signal for a graphene varactor before exposure to 1-octanethiol gold nanoparticles (C₈—S—Au NPs) is shown in plot 1302. The response signal for the same graphene varactor after modification with 1-octanethiol gold nanoparticles (C₈—S—Au NPs) is shown in plot 1304. As shown in the graph, the forward Dirac point of the graphene varactor shifts to the right from about 1.2 V (before surface modification) to about 1.8 V (after surface modification with C₈—S—Au NPs), to give a shift of approximately 0.6 V. The shift in the Dirac point for graphene modification with 1-octanethiol gold nanoparticles is indicated as arrow 1306. The difference in Dirac shift using Au NPs and C₈—S—Au NPs indicates a different doping effect of Au NPs as compared to C₈—S—Au NPs on graphene.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A medical device comprising:
a graphene varactor comprising:
a graphene layer;
at least one non-covalent modification layer disposed on an outer surface of the graphene layer; and
wherein the at least one non-covalent modification layer comprises nanoparticles selected from a group comprising one or more metals, metal oxides, or derivatives thereof.

2. The medical device of claim 1, wherein the at least one modification layer provides coverage over the graphene layer from 5% to 150% by surface area.

3. The medical device of claim 1, comprising a plurality of graphene varactors configured in an array on the medical device.

4. The medical device of claim 1, further comprising more than one non-covalent modification layer.

5. The medical device of claim 4, comprising from two to 20 distinct non-covalent modification layers.

6. The medical device of claim 4, the nanoparticles comprising metals or metal oxides selected from a group comprising gold (Au), platinum (Pt), silver (Ag), palladium (Pd), iron III oxide (Fe₂O₃), iron II, III oxide (Fe₃O₄), zinc oxide (ZnO), palladium II oxide (PdO), tin dioxide (SnO₂), titanium (Ti), titanium dioxide (TiO₂), silicon dioxide (SiO₂), cobalt diiron tetraoxide (CoFe₂O₄), indium trioxide (In₂O₃), vanadium pentoxide (V₂O₅), platinum oxide (PtO₂), copper oxide (CuO), cadmium oxide (CdO), chromium niobate (CrNbO₄), CoNb₂O₆, molybdenum disulfide (MoS₂), tungsten oxide (WO), tungsten dioxide (WO₂), tungsten trioxide (WO₃), neodymium oxide (Nd₂O₃), boron nitride (BN), CeFeO₄H, manganese oxide (Mn₃O₄), iron oxyhydroxide (FeOOH) comprising goethite, akageneite, lepidocrocite, and feroxyhyte; and any combinations, or derivatives thereof.

7. The medical device of claim 6, wherein the nanoparticles comprise gold nanoparticles.

8. The medical device of claim 1, wherein the nanoparticles are further modified with groups comprising alkylthio, alkenylthio, alkynylthio, heteroalkylthio, heteroalkenylthio, heteroalkynylthio, haloalkylthio, haloalkenylthio, haloalkynylthio, halogenated heteroalkylthio, halogenated heteroalkenylthio, halogenated heteroalkynylthio, arylthio, substituted arylthio, heteroarylthio, or substituted heteroarylthio.

9. A method of modifying a surface of graphene, the method comprising:
contacting a graphene layer with a solution or suspension comprising one or more nanoparticles comprising metals, metal oxides, or derivatives thereof;
forming at least one non-covalent modification layer of the one or more nanoparticles disposed on an outer surface of a graphene layer;
wherein the at least one non-covalent modification layer comprises one or more nanoparticles selected from a group comprising one or more metals, metal oxides, or derivatives thereof; and
quantifying an extent of surface coverage of the at least one non-covalent modification layer using contact angle goniometry, Raman spectroscopy, scanning electron microscopy (SEM), transmission electron microscopy (TEM), atomic force microscopy (AFM), scanning tunneling microscopy (STM), or X-Ray photoelectron spectroscopy.

10. The method of claim 9, wherein the at least one non-covalent modification layer provides coverage over the graphene layer from 5% to 150% by surface area.

11. The method of claim 9, wherein contacting a graphene layer with a solution or a suspension comprises immersing the graphene layer into a solution or a suspension.

12. The method of claim 9, further comprising forming more than one non-covalent modification layer.

13. The method of claim 9, the nanoparticles comprising metals or metal oxides selected from a group comprising gold (Au), platinum (Pt), silver (Ag), palladium (Pd), iron III oxide (Fe₂O₃), iron II, III oxide (Fe₃O₄), zinc oxide (ZnO), palladium II oxide (PdO), tin dioxide (SnO₂), titanium (Ti), titanium dioxide (TiO₂), silicon dioxide (SiO₂), cobalt diiron tetraoxide (CoFe₂O₄), indium trioxide (In₂O₃), vanadium pentoxide (V₂O₅), platinum oxide (PtO₂), copper oxide (CuO), cadmium oxide (CdO), chromium niobate (CrNbO₄), CoNb₂O₆, molybdenum disulfide (MoS₂), tungsten oxide (WO), tungsten dioxide (WO₂), tungsten trioxide (WO₃), neodymium oxide (Nd₂O₃), boron nitride (BN), CeFeO₄H, manganese oxide (Mn₃O₄), iron oxyhydroxide (FeOOH) comprising goethite, akageneite, lepidocrocite, and feroxyhyte; and any combinations, or derivatives thereof.

14. The method of claim 9, wherein the nanoparticles are further modified with groups comprising alkylthio, alkenylthio, alkynylthio, heteroalkylthio, heteroalkenylthio, heteroalkynylthio, haloalkylthio, haloalkenylthio, haloalkynylthio, halogenated heteroalkylthio, halogenated heteroalkenylthio, halogenated heteroalkynylthio, arylthio, substituted arylthio, heteroarylthio, or substituted heteroarylthio.

15. A method for detecting an analyte comprising:
collecting a gaseous sample;
contacting the gaseous sample with one or more graphene varactors, each of the one or more graphene varactors comprising:
a graphene layer;
at least one non-covalent modification layer disposed on an outer surface of the graphene layer; and
wherein the at least one non-covalent modification layer comprises one or more nanoparticles selected from a group comprising metals, metal oxides, or derivatives thereof.

16. The method of claim 15, the gaseous sample comprising a patient breath sample or an environmental gas sample.

17. The method of claim 15, further comprising measuring a differential response in an electrical property of the one or more graphene varactors due to binding of one or more analytes present in the gaseous sample.

18. The method of claim 16, the electrical property selected from the group consisting of capacitance or resistance.

19. The method of claim 15, the nanoparticles comprising metals or metal oxides selected from the group comprising gold (Au), platinum (Pt), silver (Ag), palladium (Pd), iron III oxide (Fe₂O₃), iron II, III oxide (Fe₃O₄), zinc oxide (ZnO), palladium II oxide (PdO), tin dioxide (SnO₂), titanium (Ti), titanium dioxide (TiO₂), silicon dioxide (SiO₂), cobalt diiron tetraoxide (CoFe₂O₄), indium trioxide (In₂O₃), vanadium pentoxide (V₂O₅), platinum oxide (PtO₂), copper oxide (CuO), cadmium oxide (CdO), chromium niobate (CrNbO₄), CoNb₂O₆, molybdenum disulfide (MoS₂), tungsten oxide (WO), tungsten dioxide (WO₂), tungsten trioxide (WO₃), neodymium oxide (Nd₂O₃), boron nitride (BN), CeFeO₄H, manganese oxide (Mn₃O₄), iron oxyhydroxide (FeOOH) comprising goethite, akageneite, lepidocrocite, and feroxyhyte; and any combinations, or derivatives thereof.

20. The method of claim 15, wherein the nanoparticles are further modified with groups comprising alkylthio, alkenylthio, alkynylthio, heteroalkylthio, heteroalkenylthio, heteroalkynylthio, haloalkylthio, haloalkenylthio, haloalkynylthio, halogenated heteroalkylthio, halogenated heteroalkenylthio, halogenated heteroalkynylthio, arylthio, substituted arylthio, heteroarylthio, or substituted heteroarylthio.
